(19) United States
(12) Patent Application Publication
(10) Pub. No.: US 2025/0304897 A1
(43) Pub. Date: Oct. 2, 2025

(54) MOVABLE LAMINATED BIOLOGICAL CULTURE DEVICE AND USING METHOD THEREOF

(71) Applicant: Qingdao Renjinli Innovation Technology Center (Sole Proprietorship), Qingdao City (CN)

(72) Inventor: Jinli Ren, Yinchuan City (CN)

(73) Assignee: Qingdao Renjinli Innovation Technology Center (Sole Proprietorship), Qingdao (CN)

(21) Appl. No.: 19/231,519

(22) Filed: Jun. 8, 2025

(30) Foreign Application Priority Data
Aug. 24, 2024 (CN) 2024111712662

(51) Int. Cl. C12M 1/00; C12M 1/32; C12M 1/34; C12M 3/00

(52) U.S. Cl. CPC C12M 23/20; C12M 23/12; C12M 23/40; C12M 23/44; C12M 23/46; C12M 41/06; C12M 41/12; C12M 41/32; C12M 41/34; C12M 47/02

(57) ABSTRACT

A movable laminated biological culture device and a using method thereof is provided. The device includes a plurality of culture space assemblies laminated up and down, a biological development factor supply part, and a biological development supply pile assembly. The culture space assembly includes a bottom part and supporting walls, the supporting walls are connected to the bottom part at edges of the bottom part in a preset manner, and the bottom part and the supporting walls are enclosed in a preset manner to form a culture space, a plurality of culture space assemblies which are laminated up and down are connected with each other through supporting walls.

### CROSS-REFERENCE TO RELATED APPLICATIONS

This application claims priority of Chinese Patent Application No. 202411171266.2, filed on Sep. 4, 2024, the contents of which is hereby incorporated by reference.

### TECHNICAL FIELD

The disclosure relates to the technical field of biological stereoscopic culture equipment for plants, mushrooms, insects and the like, in particular to a movable laminated biological culture device and a using method thereof.

### BACKGROUND

In the prior art, in order to improve the productivity of occupying land, plants, mushrooms and insect culture factories in greenhouses and other building facilities are arranged with multi-layer biological culture sites at intervals in a vertically three-dimensional laminated manner, and the multi-layer biological culture sites are supported by multi-layer lateral support structures, which are fixedly connected to column structures and supported by column structures, which are fixedly connected with the earth, and both the multi-layer lateral support structures and the column structures are immobile. This is to follow the technical idea of designing frame building structure to design three-dimensional multi-layer biological culture place.

In the prior art, in order to perform sowing or harvesting operations on multi-layer culture sites, the culture sites must be arranged in multiple rows at intervals, and operation channels with sufficient width must be reserved between each row of culture sites to perform sowing and harvesting operations on the multi-layer culture sites. The existing problems are as follows: firstly, the reserved working channel wastes the land and space in the building facilities of the biological factory; secondly, it is difficult to sow and harvest operations being performed on the culture sites in the long and narrow operation tunnel, with low operation efficiency and high operation cost.

### SUMMARY

The purpose of the disclosure is to provide a movable laminated biological culture device and a using method thereof, and to solve the above problems existing in the prior art.

In order to solve the above technical problems, the disclosure adopts the following technical scheme:

A movable laminated biological culture device is provided, and includes a plurality of culture space assemblies laminated up and down, a biological development factor supply part coordinated with each of the culture space assemblies being located in each layer, and a biological development supply pile assembly coordinated with the biological development factor supply part, where each of the culture space assemblies includes a bottom part and supporting walls, the supporting walls are connected to the bottom part at edges of the bottom part in a preset manner, and the bottom part and the supporting walls are enclosed in a preset manner to form a culture space, and the culture space is used for biological culture, and the plurality of culture space assemblies laminated up and down are detachably connected with each other through respective the supporting walls. It should be noted that the supporting walls of the culture space assemblies located at the lower part bears the gravity of all the culture space assemblies at the upper part, so the supporting walls are not only an enclosure structure of the culture space assemblies but also a load-bearing part in the culture space assemblies. The supporting walls can be composed of a load-bearing part and a sealing part, or a combination of the load-bearing part and the sealing part. A plurality of the culture space assemblies can be laminated up and down in a pile for use in a fixed position, or laminated up and down in a pile for overall mobile use, or laminated up and down together for single split mobile use. A plurality of stacks of the culture space assemblies are used close together, thus eliminating the phenomenon that a fixed aisle must be arranged in the prior art to waste space. the biological development factor supply part is arranged correspondingly to the culture space, and the biological development factor supply part includes a supporting wall clutch part and further at least includes one of a water body dispersion part, a gas dispersion part, a light emitter and a heater, where the supporting wall clutch part is correspondingly provided with at least one of a water body receiving port, a gas receiving port and a power receiving port, and the biological development factor supply part is connected with the supporting walls and/or the bottom part of the culture space in a preset manner; the biological development supply pile assembly includes a pile frame and a plurality of pile frame clutch parts arranged on the pile frame at intervals from top to bottom, where each of the pile frame clutch parts is correspondingly arranged with the biological development factor supply part, and each of the pile frame clutch parts is also correspondingly arranged with the supporting wall clutch part and at least includes one of a water body supply port, a gas supply port and a power supply port, and each of the pile frame clutch parts is detachably connected with corresponding supporting wall clutch part; the biological development supply pile assembly further includes at least one of a water body supply part, a gas supply part and a power supply part arranged corresponding to each of the pile frame clutch parts, where the water body supply part is connected with a preset water source, and the water supply part is detachably connected with the water body receiving port of the supporting wall clutch part through the water body supply port of each of the pile frame clutch parts, so as to provide or cut off moisture necessary for growth and development of organisms in the culture space through the water body dispersion part; the gas supply part is connected with a preset gas source, and the gas supply part is detachably connected with the gas receiving port of the supporting wall clutch part through the gas supply port of each of the pile frame clutch parts, so as to provide or cut off gas necessary for growth and development of the organisms in the culture space through the gas dispersion part; the power supply part is connected to a preset power supply, and the power supply part is detachably connected with the power receiving port of the supporting wall clutch part through the power supply port of each of the pile frame clutch parts, so as to provide or cut off illumination and/or temperature necessary for growth and development of the organisms in the culture space through the light emitter and/or the heater.

Further, the supporting walls are fixedly connected to the bottom part, the biological development factor supply part is fixedly connected to each of the culture space assemblies, the supporting wall clutch part is fixedly connected to the supporting walls, and the water body dispersion part, the gas dispersion part, the light emitter and the heater are fixedly connected to corresponding one of the supporting wall and/or the bottom part, each of the pile frame clutch parts is detachably connected with each of the culture space assemblies through the supporting wall clutch part. It should be noted that when the bottom part is circular or elliptical, the fixed connection between the supporting walls and the bottom part is rigid. When the bottom part is a polygon including a rectangle, the fixed connection between the corresponding supporting wall and the bottom part on each side can be a rigid connection that cannot be rotated or a hinged connection that can be rotated. The supporting walls hinged with the bottom part can rotate outward around the hinge position of the bottom part to be opened, so as to form an opening, so that the organisms cultured in the culture space can be shifted and separated from the bottom part via the opening when harvesting.

Further, the supporting walls are detachably connected to the bottom part, the biological development factor supply part is detachably connected to each of the culture space assemblies, and the supporting wall clutch part, the water body dispersion part, the gas dispersion part, the light emitter and the heater are all fixedly connected to the supporting walls; the biological development factor supply part is connected with each of the culture space assemblies along with connection of the supporting walls and the bottom part, the biological development factor supply part is separated from each of the culture space assemblies along with separation of the supporting walls and the bottom part, and each of the pile frame clutch parts is detachably connected with each of the culture space assemblies through the supporting wall clutch part.

Further, bottom ends of the supporting walls are connected to the bottom part around, and the culture space is formed by enclosure of the bottom part, the supporting walls matched with the bottom part and a previous bottom part above the bottom part, and the biological development supply pile assembly is connected with the supporting walls to supply at least one of moisture, air and power to the culture space.

Further, top ends of the supporting walls are connected to the bottom part around, and the culture space is formed by enclosure of the bottom part, the supporting walls matched with the bottom part and a next bottom part below the bottom part, and the biological development supply pile assembly is connected with the supporting walls to supply at least one of moisture, air and power to the culture space;

Further, middle parts of the supporting walls are connected to the bottom part around, and the culture space is formed by enclosure of the bottom part, the supporting walls matched with the bottom part, a previous bottom part above the bottom part and previous supporting walls above the supporting walls, and the biological development supply pile assembly is connected with the supporting walls or the previous supporting walls above the supporting walls to supply at least one of moisture, air and power to the culture space.

a structure of the bottom part is rectangular, and four supporting walls include two oppositely arranged supporting walls extending upward along the bottom part and two oppositely arranged supporting walls extending downward along the bottom part; the culture space is formed by enclosure of the bottom part, the two supporting walls extending upward along the bottom part, a previous bottom part above the bottom part and two supporting walls extending downward from the previous bottom part, and the biological development supply pile assembly is connected with the supporting walls extending upward along the bottom part or the supporting walls extending downward along the bottom part.

the structure of the bottom part is rectangular, and the supporting walls include two slideway walls arranged oppositely, where a top and a bottom of each of the slideway walls are respectively provided with a slide rail part and a sliding part matched with each other up and down, and an upper layer culture space assembly is capable of sliding away from a lower layer culture space assembly along the slide rail part or the sliding part on each of the slideway walls, and the biological development supply pile assembly is connected with one of the supporting walls on at least one of four sides of the bottom part.

Further, other two supporting walls arranged vertically opposite to the two slideway walls are harvesting walls, and a length of each of the harvesting walls extending downwards along the bottom part is less than that of each of the slideway walls extending downwards along the bottom part, so that the upper layer culture space assembly slides away from the lower layer culture space assembly along the slide rail part or the sliding part on each of the slideway walls, meanwhile preset positions of organisms cultured in the lower layer culture space assembly are harvested and pushed away in a same direction by means of one of the harvesting walls of the upper layer culture space assembly, so as to realize harvesting of the organisms, and the biological development supply pile assembly is connected with at least one of the supporting walls of each of the slideway walls and/or each of the harvesting walls.

Further, the structure of the bottom part is rectangular, the supporting walls includes two slideway walls arranged oppositely, a top and a bottom of each of the slideway walls are respectively provided with a slide rail part and a sliding part matched with each other up and down, and upper and lower parts of the bottom part corresponding to the slideway walls are respectively provided with a slide rail part and a sliding part matched with each of the slideway walls up and down, the bottom part is capable of sliding away from lower supporting walls along the slide rail part or the sliding part on each of the slideway walls, and the supporting walls are capable of sliding away from a lower bottom part along the slide rail part or the sliding part on the bottom part, and the biological development supply pile assembly is connected with one of the supporting walls on at least one of four sides of the bottom part.

Further, the bottom part includes a plurality of pits arranged downwards, the pits are used for bearing nutrient substrates needed for cultivating preset organisms, so that the preset organisms absorb nutrients from the nutrient substrates in the pits, and the biological development supply pile assembly is also used for supplying at least one of moisture and air to the nutrient substrates in the pits, besides supplying air and moisture to the organisms cultured in the culture space.

A using method for movable laminated biological culture devices is provided, and includes movable laminated biological culture devices and buildings. each of the buildings includes a beam-column part, and a supply pile connection part is arranged on the beam-column part, and each of the movable laminated biological culture devices is arranged in corresponding one of the buildings for use, and used close to the beam-column part, and the supporting wall clutch part is opposite to the supply pile connection part on the beam-column part. The biological development supply pile assembly is arranged at a position close to the supply pile connection part; the biological development supply pile assembly is connected or separated from the beam-column part through the supply pile connection part; before the culture space assembly is activated, the biological development supply pile assembly is connected with the beam-column part through the supply pile connection part; further, at least one of the preset water source, the preset gas source and the preset power supply is connected with the beam-column part and is connected with the biological development supply pile assembly along the beam-column part; and where the using method at least includes:
when at least one of moisture, gas, or power needs to be supplied to culture space of each of the movable laminated biological culture devices, separating the biological development supply pile assembly from the beam-column part, and connecting each of the pile frame clutch parts of the biological development supply pile assembly with the culture space assembly through the supporting wall clutch part;
when the culture space assembly of each of the movable laminated biological culture devices needs to be transported and moved, separating each of the pile frame clutch parts of the biological development supply pile assembly from the supporting wall clutch part, so that the biological development supply pile assembly is separated from the culture space assembly, and then connecting the biological development supply pile assembly with the beam-column part through the supply pile connection part.

It should be noted that the operation that the biological development supply pile assembly reciprocates between the supply pile connection part and the supporting wall clutch part and alternately connects and separates with the supply pile connection part and the supporting wall clutch part can be performed manually or by a special actuator. When it is executed by a special actuator, the preferred scheme is to provide a reciprocating guide rail, which is fixedly connected to the beam-column part and/or the earth between the supply pile connection part and the supporting wall clutch part. A reciprocating mechanism matched with the reciprocating guide rail is provided at the position of the pile frame corresponding to the reciprocating guide rail, and the pile frame bears the pile frame clutch part to move between the supply pile connection part and the supporting wall clutch part on the reciprocating guide rail by means of the reciprocating mechanism, so that the pile frame clutch part is butted with the supply pile connection part or the supporting wall clutch part. The power modes for driving the pile frame to reciprocate at least include: firstly, setting telescopic mechanisms (such as pneumatic rods, hydraulic rods, worm gears and other mechanisms) to push and pull the pile frame; secondly, a driving mechanism is arranged on the reciprocating mechanism body; thirdly, pushing and pulling the pile frame manually.

A using method for movable laminated biological culture devices is provided, and includes movable laminated biological culture devices, the movable laminated biological culture devices are used for cultivation of organisms, and a process from sowing biological seeds to harvesting organisms at least includes:
S1, biological culture process
S11, sowing biological seeds to a bottom part of an upper part first-layer culture space assembly to be seeded in each of the movable laminated biological culture devices, and then suspending a seeded culture space assembly upward to be separated from the upper part second-layer culture space assembly and then transferring to a preset bracket, so as to become a culture space assembly being about to enter the biological culture process in the lower part first-layer of the preset bracket;
S12, continuously sowing biological seeds to a bottom part of an upper part second-layer culture space assembly to be seeded in each of the movable laminated biological culture devices, and then suspending a seeded culture space assembly upward to be separated from an upper part third-layer culture space assembly, and then transferring to above a lower part first-layer culture space assembly, being about to enter the biological culture process, of the preset bracket, so as to become a lower part second-layer culture space assembly, being about to enter the biological culture process, of the preset bracket;
S13, continuously sowing biological seeds to a bottom part of an upper part third-layer culture space assembly to be seeded in each of the movable laminated biological culture devices, then suspending a seeded culture space assembly upward to be separated from an upper part fourth-layer culture space assembly, and then transferring to above a lower part second-layer culture space assembly, being about to enter the biological culture process, of the preset bracket, so as to become a a lower part third-layer culture space assembly, being about to enter the biological culture process in, of the preset bracket;
S14, by analogy, until completing sowing biological seeds of each layer of culture space assembly, and connecting the biological development supply pile assembly with a seeded culture space assembly in each layer on the preset bracket, so as to perform cultivation of organisms, until the cultivation of organisms is completed, and entering harvesting organisms process;
S2, harvesting organisms process
S21, separating the biological development supply pile assembly from a culture space assembly being completed cultivation of organisms on each layer of the preset bracket;
S22, harvesting organisms in an upper part first-layer culture space assembly to be harvested in each of the movable laminated biological culture devices on the preset bracket, and then suspending a harvested culture space assembly upward to be separated from the upper part second-layer culture space assembly and transferring to a preset device;
it should be noted that the preset device can be a preset bracket, can be a preset conveyor belt, can also be a preset vehicle, etc., and the disclosure is not limited (the same below);
S23, continuously harvesting organisms in an upper part second-layer culture space assembly to be harvested of each of the movable laminated biological culture devices, and then suspending a harvested culture space assembly upward to be separated from the upper part third-layer culture space assembly, and then transferring to a preset device;
S24, continuously harvesting organisms in an upper part third-layer culture space assembly to be harvested in each of the movable laminated biological culture devices, and then suspending a harvested culture space assembly upward to be separated from an upper part fourth-layer culture space assembly and transferring to a preset device;
S25, by analogy, until completing harvesting of organisms in each layer of culture space assembly.

A using method for movable laminated biological culture devices is provided, and includes movable laminated biological culture devices, the movable laminated biological culture devices are used for cultivation of organisms, and a process from sowing biological seeds to harvesting organisms at least include:
S1, biological culture process
S11, sowing biological seeds to a bottom part of an upper part first-layer culture space assembly to be seeded in each of the movable laminated biological culture devices, and then pushing and transferring an upper part first-layer culture space assembly having been seeded to the preset bracket by means of the slide rail part and the sliding part on each of the slideway walls, to become a lower part first-layer culture space assembly, being about to enter the biological culture process, of the preset bracket;
S12, continuously sowing biological seeds to a bottom part of an upper part second-layer culture space assembly to be seeded in each of the movable laminated biological culture devices, and then continuously pushing and transferring an upper part second-layer culture space assembly having been seeded to above a lower part first-layer culture space assembly, being about to enter the biological culture process, of the preset bracket by means of the slide rail part and the sliding part on each of the slideway walls, so as to become a lower part second-layer culture space assembly, being about to enter the biological culture process, of the preset bracket;
S13, continuously sowing biological seeds to a bottom part of an upper part third-layer culture space assembly to be seeded in each of the movable laminated biological culture devices, and then continuously pushing and transferring an upper part third-layer culture space assembly having been seeded to above a lower part second-layer culture space assembly, being about to enter the biological culture process, of the preset bracket by means of the slide rail part and sliding part on each of the slideway walls, so as to become a lower part third-layer culture space assembly, being about to enter the biological culture process, of the preset bracket;
S14, by analogy, until completing sowing biological seeds of each layer of culture space assembly, and connecting the biological development supply pile assembly with a seeded culture space assembly in each layer on the preset bracket, so as to perform cultivation of organisms until the cultivation of organisms is completed, and entering harvesting organisms process;
S2, harvesting organisms process
S21, separating the biological development supply pile assembly from a culture space assembly being completed cultivation of organisms on each layer of the preset bracket;
S22, harvesting organisms in an upper part first-layer culture space assembly to be harvested in each of the movable laminated biological culture devices on the preset bracket, then pushing and transferring a harvested upper part first-layer culture space assembly to a preset device by means of the slide rail part and the sliding part on each of the slideway walls;
S23. continuously harvesting organisms in an upper part second-layer culture space assembly to be harvested of each of the movable laminated biological culture devices, and then continuously pushing and transferring a harvested upper part second-layer culture space assembly to a preset device by means of the slide rail part and sliding part on each of the slideway walls;
S24, continuously harvesting organisms in an upper part third-layer culture space assembly to be harvested in each of the movable laminated biological culture devices, and then continuously pushing and transferring a harvested upper part third-layer culture space assembly to a preset device by means of the slide rail part and sliding part on each of the slideway walls;
S25, by analogy, until completing harvesting of organisms in each layer of culture space assembly.

A using method for movable laminated biological culture devices is provided, and includes movable laminated biological culture devices, and applying the movable laminated biological culture devices to a joint operation of harvesting organisms to sowing biological seeds at least includes:
S1, separating the biological development supply pile assembly from a culture space assembly of each layer having been completed cultivation of organisms;
S2, harvesting organisms in an upper part first-layer culture space assembly to be harvested in each of the movable laminated biological culture devices, then purifying a bottom part of a first-layer culture space assembly having completed harvesting organisms and sowing biological seeds, and transferring a seeded culture space assembly to a preset bracket to become a lower part first-layer culture space assembly, being about to enter the biological culture process again, of the preset bracket;
S3, harvesting organisms in an upper part second-layer culture space assembly to be harvested in each of the movable laminated biological culture devices, then purifying a bottom part of a second-layer culture space assembly having completed harvesting organisms and sowing biological seeds, and transferring a seeded culture space assembly to an upper part of a lower part first-layer culture space assembly, being about to enter the biological culture process again, of the preset bracket, so as to become the lower part second-layer culture space assembly, being about to enter the biological culture process again, of the preset bracket;
S4, harvesting organisms in an upper part third-layer culture space assembly to be harvested in each of the movable laminated biological culture devices, then purifying a bottom part of a third-layer culture space assembly having completed harvesting organisms and sowing biological seeds, and transferring a seeded culture space assembly to an upper part of a lower part second-layer culture space assembly, being about to enter the biological culture process again, of the preset bracket, so as to become a lower part third-layer culture space assembly, being about to enter the biological culture process again, of the preset bracket;
S5, by analogy, simultaneously completing harvesting organisms, purification treatment and sowing biological seeds for each layer of culture space assembly through joint operation, and connecting the biological development supply pile assembly with a culture space assembly having been seeded in each layer on the preset bracket, so as to continue cultivation of organisms until the cultivation of organisms is completed, and then re-circulating into a joint operation process of harvesting organisms, synchronous purification treatment and sowing.

A using method for a movable laminated biological culture device is provided, and includes movable laminated biological culture devices, and applying the movable laminated biological culture devices to product mushroom at least includes:
S1, separating the biological development supply pile assembly in each of the movable laminated biological culture devices from each layer of culture space assembly;
S2, separating a bottom part and supporting walls in an upper part first-layer culture space assembly in each of the movable laminated biological culture devices, simultaneously arranging mushroom culture substrate on an upper part first-layer bottom part, and transferring a bottom part bearing the mushroom culture substrate to a preset bracket to become a lower part first-layer bottom part, being about to enter a substrate disinfection process, of the preset bracket;
S3, separating a bottom part and supporting walls in an upper part second-layer culture space assembly in each of the movable laminated biological culture devices, and simultaneously arranging the mushroom culture substrate on an upper part second-layer bottom part, and transferring a bottom part bearing the mushroom culture substrate to a lower part first-layer bottom part of the preset bracket to become a lower part second-layer bottom part, being about to enter the substrate disinfection process, of the preset bracket;
S4, separating a bottom part and supporting walls in an upper part third-layer culture space assembly in each of the movable laminated biological culture devices, and simultaneously arranging the mushroom culture substrate on an upper part third-layer bottom part, and transferring a bottom part bearing the mushroom culture substrate to a lower part second-layer bottom part on the preset bracket to become a lower part third-layer bottom part, being about to enter the substrate disinfection process, of the preset bracket;
S5, by analogy, separating a bottom part and supporting walls in each layer of culture space assembly in each of the movable laminated biological culture devices, and completing arrangement of mushroom culture substrate on each layer of a bottom part, and overlapping a bottom part, bearing the mushroom culture substrate, of each layer on a preset bracket;
S6, sterilizing a bottom part of each layer on the preset bracket together with bore mushroom culture substrate;
S7, performing mushroom inoculation on mushroom culture substrate on an upper part first-layer bottom part having been disinfected on the preset bracket, transferring a bottom part having been completed mushroom inoculation to a new preset bracket to become a lower part first-layer bottom part of the new preset bracket, and simultaneously arranging one of the supporting walls on the lower part first-layer bottom part to form lower part first-layer culture space of the new preset bracket, and arranging the biological development factor supply part in the lower part first-layer culture space together with the supporting wall;
S8, performing mushroom inoculation on an upper part second-layer bottom part having been disinfected on the preset bracket, transferring a bottom part having been inoculated with mushroom to a supporting wall on the lower part first-layer bottom part of the new preset bracket, and simultaneously arranging another supporting wall on the lower part first-layer bottom part to form a lower part second-layer culture space of the new preset bracket, and arranging the biological development factor supply part in the lower part second-layer culture space together with the supporting wall;
S9, performing mushroom inoculation on an upper part third-layer bottom part having been disinfected on the preset bracket, transferring a bottom part having been inoculated with mushroom to a supporting wall on a lower part second-layer bottom part of the new preset bracket, and simultaneously arranging another supporting wall on the lower part second-layer bottom part to form a lower part third-layer culture space of the new preset bracket, and arranging the biological development factor supply part in the lower part third-layer culture space together with the supporting wall;
S10, by analogy, until completing inoculation of mushroom culture substrate on a bottom part of each layer, and re-forming laminated culture space, and then connecting the biological development supply pile assembly with a culture space assembly of each layer, and entering mushroom culture process until mushroom culture is successful, and entering mushroom harvesting process;
S11, separating the biological development supply pile assembly from each layer of culture space assembly;
S12, harvesting mushroom in each culture space at an upper part of the new preset bracket, and separating supporting walls and a bottom part of each layer for later use.

Compared with the prior art, the beneficial technical effects of the disclosure are as follows:

The movable laminated biological culture device and the using method thereof in the disclosure, in the process of three-dimensional multi-layer culture of industrialized plants, mushrooms, insects and other organisms, by arranging a plurality of biological development factor supply parts matched with each layer of culture space and a biological development supply pile assembly matched with each biological development factor supply part, a stack of multi-layer laminated culture space assemblies can be used together with another stack of multi-layer laminated culture space assemblies, and there is no need to set an operation channel, thus solving the problem that the reserved operation channel wastes land and space in the building facilities of the biological factory in the prior art. At the same time, because the biological culture space is movable, the biological seeds sowing operation, the biological culture operation and the biological harvesting operation can be flexibly realized by moving the biological culture space. Not only can each layer of a stack of multi-layer laminated culture space assemblies be separately transferred to a preset site to perform biological seeds sowing operation, biological culture operation and biological harvesting operation, but also a stack of multi-layer laminated culture space assemblies can be transferred to a preset site as a whole to perform biological seeds sowing operation, biological culture operation and biological harvesting operation on each layer of culture space respectively. Thereby the problems of great difficulty, low operating efficiency and high operating cost in seeding and harvesting cultivation sites in the prior art are solved. The disclosure has reasonable overall structural design and convenient use, and is suitable for popularization and application.

### BRIEF DESCRIPTION OF THE DRAWINGS

In order to explain the embodiments of the disclosure or the technical scheme in the prior art more clearly, the drawings needed in the embodiments will be briefly introduced below. Obviously, the drawings in the following description are only some embodiments of the disclosure, and other drawings can be obtained according to these drawings without creative efforts for ordinary skilled in the art.

FIG. 1 is a schematic structural diagram of a movable laminated biological culture device in a front view according to Embodiment 1 of the disclosure;

FIG. 2 is a schematic diagram of the three-dimensional structure of the movable laminated biological culture device according to Embodiment 1 of the disclosure;

FIG. 3 is a schematic structural diagram of the movable laminated biological culture device in a top view according to Embodiment 1 of the disclosure;

FIG. 4 is a schematic structural diagram of the movable laminated biological culture device in a top view when the bottom structure is provided with a heater according to Embodiment 1 of the disclosure;

FIG. 5 is a schematic structural diagram of the movable laminated biological culture device when the supporting wall is provided with a relocation structure according to Embodiment 1 of the disclosure;

FIG. 6 is a schematic structural diagram of a movable laminated biological culture device in a front view according to Embodiment 2 of the disclosure;

FIG. 7 is a schematic structural diagram of the movable laminated biological culture device according to Embodiment 2 of the disclosure, showing that both the supporting wall and the bottom structure are provided with a sliding rail structure and a sliding structure;

FIG. 8 is a schematic structural diagram of a movable laminated biological culture device in a front view according to Embodiment 3 of the disclosure;

FIG. 9 is a schematic structural diagram of the movable laminated biological culture device according to Embodiment 3 of the disclosure, showing that the supporting wall clutch part and the pile frame clutch part are separated;

FIG. 10 is a schematic structural diagram of the movable laminated biological culture device according to Embodiment 3 of the disclosure, showing that the bottom structure includes a plurality of downward pits;

FIG. 11 is a schematic structural diagram of the movable laminated biological culture device according to Embodiment 3 of the disclosure, showing that the supporting wall includes two slideway walls arranged oppositely;

FIG. 12 is a schematic structural diagram of the movable laminated biological culture device according to Embodiment 3 of the disclosure, showing that the bottom structure is provided with a heater;

FIG. 13 is a schematic diagram of the three-dimensional structure of a movable laminated biological culture device according to Embodiment 4 of the disclosure;

FIG. 14 is a schematic diagram of the three-dimensional structure of a movable laminated biological culture device according to Embodiment 5 of the disclosure;

FIG. 15 is a schematic structural diagram of a movable laminated biological culture device according to Embodiment 6 of the disclosure, showing that the supporting wall includes two slideway walls arranged oppositely;

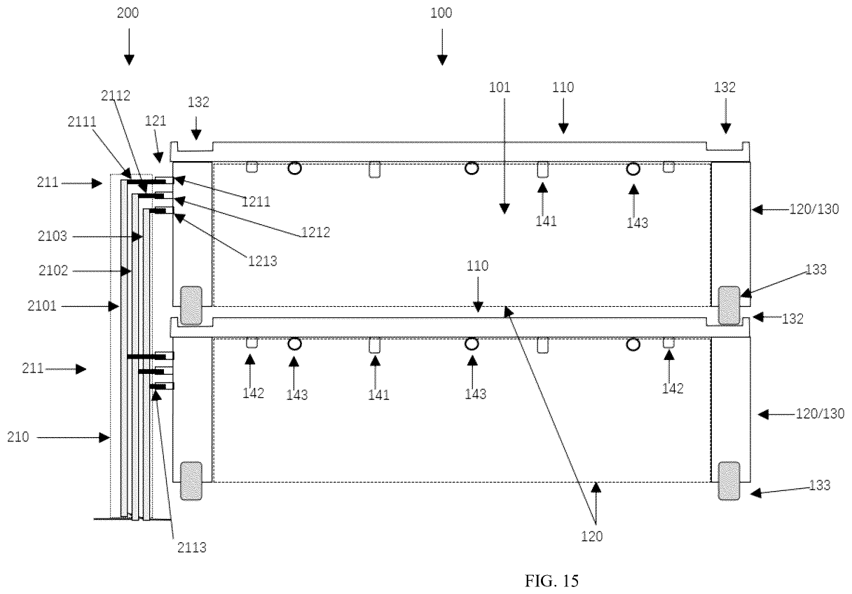

FIG. 16 is a schematic structural diagram of the movable laminated biological culture device according to Embodiment 6 of the disclosure, showing that the supporting wall includes two harvesting walls arranged vertically to the slideway wall; and

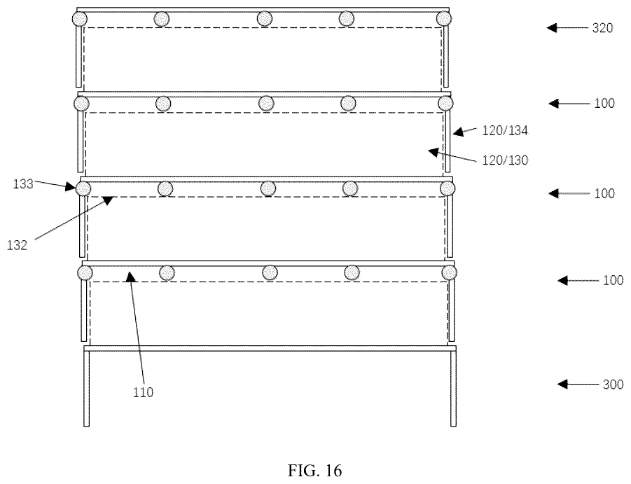

FIG. 17 is a schematic structural diagram of the using method of a movable laminated biological culture device used in the building according to Embodiment 7 of the disclosure.

List of reference characters: 1 movable laminated biological culture device; 2 preset water source; 3 preset gas source; 4 preset power supply; 5 multi-span greenhouse; 6 beam-column structure; 7 mushroom culture substrate; 8 mushroom; 100 culture space assembly; 101 culture space; 102 air flow cavity; 110 bottom structure; 111 pit; 120 supporting wall; 121 supporting wall clutch part; 1211 water body receiving port; 1212 gas receiving port; 1213 power receiving port; 122 supporting wall clutch part coupler; 123 relocation structure; 130 slideway wall; 131 clutch supporting wall; 132 slide rail structure; 133 sliding structure; 134 harvesting wall; 135 reinforcing structure; 140 biological development factor supply part; 141 water body dispersion structure; 142 gas dispersion structure; 143 light emitter; 144 heater; 145 lamp holder; 146 sprayer; 147 sterilization lamp; 200 biological development supply pile assembly; 201 booster pump; 202 air flow pump; 2021 air flow; 210 pile frame; 2101 water body supply part; 2102 gas supply part; 2103 power supply part; 211 pile frame clutch part; 2111 water body supply port; 2112 gas supply port; 2113 power supply port; 212 pile frame clutch part stabilizer; 300 bottom supporting assembly; 310 supply pile connection part; 320 top cover assembly; 321 disk cover slide rail part, 322 disk cover sliding structure; and 323 bracket slide rail structure.

### DETAILED DESCRIPTION OF THE EMBODIMENTS

In the following, embodiments of the disclosure will be described in detail, examples of which are illustrated in the accompanying drawings, where the same or similar reference numerals indicate the same or similar elements or elements having the same or similar functions throughout. The embodiments described below by referring to the drawings are exemplary, only for explaining the disclosure, and cannot be understood as limiting the disclosure.

In the description of this disclosure, it should be understood that the azimuth or positional relationship indicated by the terms “length”, “width”, “inside” and “outside” is based on the azimuth or positional relationship shown in the attached drawings, and is only for the convenience of describing this disclosure and simplifying the description, and does not indicate or imply that the device or element referred to must have a specific orientation, be constructed and operated in a specific orientation, so it cannot be understood as a limitation of this disclosure.

In the description of this disclosure, “multiple” means two or more, unless otherwise specified; the “preset bracket” is a device used in cooperation with the culture space assembly, which can be lifted by itself or by lifting equipment to bear the culture space assembly in a proper posture.

In the description of this disclosure, it should be noted that unless otherwise specified and limited, the terms “installed”, “connected” and “connection” should be broadly understood, for example, they can be fixed, detachable or integrally connected; they can be directly connected, can also be indirectly connected through an intermediary, can be the internal connection of two elements or the interaction relation between two elements. For those skilled in the art, the specific meanings of the above terms in this disclosure can be understood according to specific circumstances.

The embodiment of the disclosure provides a movable laminated biological culture device, which includes a plurality of culture space assemblies laminated up and down, a biological development factor supply part coordinated with each of the culture space assemblies being located in each layer, and a biological development supply pile assembly coordinated with the biological development factor supply part, where each of the culture space assemblies includes a bottom part and supporting walls, the supporting walls are connected to the bottom part at edges of the bottom part in a preset manner, and the bottom part and the supporting walls are enclosed in a preset manner to form a culture space, and the culture space is used for biological culture, and the plurality of culture space assemblies laminated up and down are detachably connected with each other through respective the supporting walls. A separable gravity supporting structure is formed from top to bottom, and the supporting wall at the lower part bears the gravity of all the culture space assemblies above the body. The biological development factor supply part is arranged correspondingly to the culture space, and the biological development factor supply part includes a supporting wall clutch part and further at least includes one of a water body dispersion part, a gas dispersion part, a light emitter and a heater. The specific composition of the biological development factor sub-supply part arranged in the culture space assembly for different kinds of biological culture can be different, for example, most plant cultures need to be provided with water body dispersion structure, gas dispersion structure and light emitter, while some insect cultures need to be provided with water body dispersion structure, gas dispersion structure and nutrient dispersion structure, etc. The supporting wall clutch part is correspondingly provided with at least one of a water body receiving port, a gas receiving port and a power receiving port, and the biological development factor supply part is connected with the supporting walls and/or the bottom part of the culture space in a preset manner; the biological development supply pile assembly includes a pile frame and a plurality of pile frame clutch parts arranged on the pile frame at intervals from top to bottom, where each of the pile frame clutch parts is correspondingly arranged with the biological development factor supply part, and each of the pile frame clutch parts is also correspondingly arranged with the supporting wall clutch part and at least includes one of a water body supply port, a gas supply port and a power supply port, and each of the pile frame clutch parts is detachably connected with corresponding supporting wall clutch part; the biological development supply pile assembly further includes at least one of a water body supply part, a gas supply part and a power supply part arranged corresponding to each of the pile frame clutch parts, where the water body supply part is connected with a preset water source, and the water supply part is detachably connected with the water body receiving port of the supporting wall clutch part through the water body supply port of each of the pile frame clutch parts, so as to provide or cut off moisture necessary for growth and development of organisms in the culture space through the water body dispersion part; the gas supply part is connected with a preset gas source, and the gas supply part is detachably connected with the gas receiving port of the supporting wall clutch part through the gas supply port of each of the pile frame clutch parts, so as to provide or cut off gas necessary for growth and development of the organisms in the culture space through the gas dispersion part; the power supply part is connected to a preset power supply, and the power supply part is detachably connected with the power receiving port of the supporting wall clutch part through the power supply port of each of the pile frame clutch parts, so as to provide or cut off illumination and/or temperature necessary for growth and development of the organisms in the culture space through the light emitter and/or the heater.

Optionally, the supporting walls are fixedly connected to the bottom part, the biological development factor supply part is fixedly connected to each of the culture space assemblies, the supporting wall clutch part is fixedly connected to the supporting walls, and the water body dispersion part, the gas dispersion part, the light emitter and the heater are fixedly connected to corresponding one of the supporting wall and/or the bottom part, each of the pile frame clutch parts is detachably connected with each of the culture space assemblies through the supporting wall clutch part.

Optionally, the supporting walls are detachably connected to the bottom part, the biological development factor supply part is detachably connected to each of the culture space assemblies, and the supporting wall clutch part, the water body dispersion part, the gas dispersion part, the light emitter and the heater are all fixedly connected to the supporting walls; the biological development factor supply part is connected with each of the culture space assemblies along with connection of the supporting walls and the bottom part, the biological development factor supply part is separated from each of the culture space assemblies along with separation of the supporting walls and the bottom part, and each of the pile frame clutch parts is detachably connected with each of the culture space assemblies through the supporting wall clutch part. The advantage of this scheme is that when the culture space assembly is used for mushroom culture, there is a link to sterilize the mushroom culture substrate at high temperature, and during the operation of this link, the supporting wall carried with the biological development factor supply part is separated from the bottom structure carried with the mushroom culture substrate, so as to avoid the damage of high temperature environment to the biological development factor supply part.

Optionally, bottom ends of the supporting walls are connected to the bottom part around, and the culture space is formed by enclosure of the bottom part, the supporting walls matched with the bottom part and a previous bottom part above the bottom part, and the biological development supply pile assembly is connected with the supporting walls to supply at least one of moisture, air and power to the culture space.

Optionally, top ends of the supporting walls are connected to the bottom part around, and the culture space is formed by enclosure of the bottom part, the supporting walls matched with the bottom part and a next bottom part below the bottom part, and the biological development supply pile assembly is connected with the supporting walls to supply at least one of moisture, air and power to the culture space.

Optionally, middle parts of the supporting walls are connected to the bottom part around, and the culture space is formed by enclosure of the bottom part, the supporting walls matched with the bottom part, a previous bottom part above the bottom part and previous supporting walls above the supporting walls, and the biological development supply pile assembly is connected with the supporting walls or the previous supporting walls above the supporting walls to supply at least one of moisture, air and power to the culture space.

Optionally, a structure of the bottom part is rectangular, and four supporting walls include two oppositely arranged supporting walls extending upward along the bottom part and two oppositely arranged supporting walls extending downward along the bottom part; the culture space is formed by enclosure of the bottom part, the two supporting walls extending upward along the bottom part, a previous bottom part above the bottom part and two supporting walls extending downward from the previous bottom part, and the biological development supply pile assembly is connected with the supporting walls extending upward along the bottom part or the supporting walls extending downward along the bottom part.

Optionally, the structure of the bottom part is rectangular, and the supporting walls include two slideway walls arranged oppositely, where a top and a bottom of each of the slideway walls are respectively provided with a slide rail part and a sliding part matched with each other up and down, and an upper layer culture space assembly is capable of sliding away from a lower layer culture space assembly along the slide rail part or the sliding part on each of the slideway walls, and the biological development supply pile assembly is connected with one of the supporting walls on at least one of four sides of the bottom part.

Optionally, other two supporting walls arranged vertically opposite to the two slideway walls are harvesting walls, and a length of each of the harvesting walls extending downwards along the bottom part is less than that of each of the slideway walls extending downwards along the bottom part, so that the upper layer culture space assembly slides away from the lower layer culture space assembly along the slide rail part or the sliding part on each of the slideway walls, meanwhile preset positions of organisms cultured in the lower layer culture space assembly are harvested and pushed away in a same direction by means of one of the harvesting walls of the upper layer culture space assembly, so as to realize harvesting of the organisms, and the biological development supply pile assembly is connected with at least one of the supporting walls of each of the slideway walls and/or each of the harvesting walls.

Optionally, the structure of the bottom part is rectangular, the supporting walls includes two slideway walls arranged oppositely, a top and a bottom of each of the slideway walls are respectively provided with a slide rail part and a sliding part matched with each other up and down, and upper and lower parts of the bottom part corresponding to the slideway walls are respectively provided with a slide rail part and a sliding part matched with each of the slideway walls up and down, the bottom part is capable of sliding away from lower supporting walls along the slide rail part or the sliding part on each of the slideway walls, and the supporting walls are capable of sliding away from a lower bottom part along the slide rail part or the sliding part on the bottom part, and the biological development supply pile assembly is connected with one of the supporting walls on at least one of four sides of the bottom part.

Optionally, the bottom part includes a plurality of pits arranged downwards, the pits are used for bearing nutrient substrates needed for cultivating preset organisms, so that the preset organisms absorb nutrients from the nutrient substrates in the pits, and the biological development supply pile assembly is also used for supplying at least one of moisture and air to the nutrient substrates in the pits.

The embodiment of the disclosure also provides a using method for movable laminated biological culture devices, which includes movable laminated biological culture devices and buildings, where each of the buildings includes a beam-column part, and a supply pile connection part is arranged on the beam-column part, and each of the movable laminated biological culture devices is arranged in corresponding one of the buildings for use, and close to supply pile connection part, so that the biological development supply pile assembly is arranged at a position close to the supply pile connection part; the biological development supply pile assembly is connected or separated from the beam-column part through the supply pile connection part; before the culture space assembly is activated, the biological development supply pile assembly is connected with the beam-column part through the supply pile connection part; further, at least one of the preset water source, the preset gas source and the preset power supply is connected with the beam-column part and is connected with the biological development supply pile assembly along the beam-column part; and the using method at least includes:
when at least one of moisture, gas, or power needs to be supplied to culture space of each of the movable laminated biological culture devices, the biological development supply pile assembly is separated from the beam-column part, and each of the pile frame clutch parts of the biological development supply pile assembly is connected with the culture space assembly through the supporting wall clutch part;
when the culture space assembly of each of the movable laminated biological culture devices needs to be transported and moved, each of the pile frame clutch parts of the biological development supply pile assembly is separated from the supporting wall clutch part, so that the biological development supply pile assembly is separated from the culture space assembly, and then the biological development supply pile assembly is connected with the beam-column part through the supply pile connection part.

The embodiment of the disclosure also provides a using method for movable laminated biological culture devices, which includes movable laminated biological culture devices, the movable laminated biological culture devices are used for cultivation of organisms, and a process from sowing biological seeds to harvesting organisms at least includes:
S1, biological culture process
S11, biological seeds are sowed to a bottom part of an upper part first-layer culture space assembly to be seeded in each of the movable laminated biological culture devices, and then a seeded culture space assembly is suspended upward to be separated from the upper part second-layer culture space assembly and then it is transferred to a preset bracket, so as to become a lower part first-layer culture space assembly, being about to enter the biological culture process, of the preset bracket;
S12, biological seeds are continuously sowed to a bottom part of an upper part second-layer culture space assembly to be seeded in each of the movable laminated biological culture devices, and then a seeded culture space assembly is suspended upward to be separated from an upper part third-layer culture space assembly, and then it is transferred to above a lower part first-layer culture space assembly, being about to enter the biological culture process, of the preset bracket, so as to become a lower part second-layer culture space assembly being about to enter the biological culture process, of the preset bracket;
S13, biological seeds are continuously sowed to a bottom part of an upper part third-layer culture space assembly to be seeded in each of the movable laminated biological culture devices, then a seeded culture space assembly is suspended upward to be separated from an upper part fourth-layer culture space assembly, and then transferring to above a lower part second-layer culture space assembly, being about to enter the biological culture process, of the preset bracket, so as to become a lower part third-layer culture space assembly, being about to enter the biological culture process, of the preset bracket;
S14, by analogy, until sowing biological seeds of each layer of culture space assembly is completed, and the biological development supply pile assembly is connected with a seeded culture space assembly in each layer on the preset bracket, so as to perform cultivation of organisms, until the cultivation of organisms is completed, and entering harvesting organisms process;
S2, harvesting organisms process
S21, the biological development supply pile assembly is separated from a culture space assembly being completed cultivation of organisms on each layer of the preset bracket;
S22, organisms in an upper part first-layer culture space assembly to be harvested in each of the movable laminated biological culture devices on the preset bracket are harvested, and then a harvested culture space assembly is suspended upward to be separated from the upper part second-layer culture space assembly and transferred to a preset device;
S23, organisms are continuously harvested in an upper part second-layer culture space assembly to be harvested of each of the movable laminated biological culture devices, and then a harvested culture space assembly is suspended upward to be separated from the upper part third-layer culture space assembly, and then transferred to a preset device;
S24, organisms are continuously harvested in an upper part third-layer culture space assembly to be harvested in each of the movable laminated biological culture devices, and then a harvested culture space assembly is suspended upward to be separated from an upper part fourth-layer culture space assembly and transferred to a preset device;
S25, by analogy, until harvesting of organisms in each layer of culture space assembly is completed.

The embodiment of the disclosure also provides a using method for movable laminated biological culture devices, which includes movable laminated biological culture devices, the movable laminated biological culture devices are used for cultivation of organisms, and a process from sowing biological seeds to harvesting organisms at least includes:
S1, biological culture process
S11, biological seeds are sowed to a bottom part of an upper part first-layer culture space assembly to be seeded in each of the movable laminated biological culture devices, and then an upper part first-layer culture space assembly having been seeded is pushed and transferred to the preset bracket by means of the slide rail part and the sliding part on each of the slideway walls, to become a lower part first-layer culture space assembly, being about to enter the biological culture process, of the preset bracket; it should be noted that the preset bracket in this scheme can be lifted and lowered, and its real-time height is matched with the culture space assemblies in a stack of movable laminated biological culture devices to be operated, so as to facilitate to bear the culture space assemblies through the slide rail structure (the same below);
S12, biological seeds are continuously sowed to a bottom part of an upper part second-layer culture space assembly to be seeded in each of the movable laminated biological culture devices, and then an upper part second-layer culture space assembly having been seeded is continuously pushed and transferred to above a lower part first-layer culture space assembly, being about to enter the biological culture process, of the preset bracket by means of the slide rail part and the sliding part on each of the slideway walls, so as to become a a lower part second-layer culture space assembly, being about to enter the biological culture process, of the preset bracket;
S13, biological seeds are continuously sowed to a bottom part of an upper part third-layer culture space assembly to be seeded in each of the movable laminated biological culture devices, and then an upper part third-layer culture space assembly having been seeded is continuously pushed and transferred to above a lower part second-layer culture space assembly, being about to enter the biological culture process, of the preset bracket by means of the slide rail part and sliding part on each of the slideway walls, so as to become a lower part third-layer culture space assembly, being about to enter the biological culture process, of the preset bracket;
S14, by analogy, until sowing biological seeds of each layer of culture space assembly is completed, and the biological development supply pile assembly is connected with a seeded culture space assembly in each layer on the preset bracket, so as to perform cultivation of organisms until the cultivation of organisms is completed, and harvesting organisms process is entered;
S2, harvesting organisms process
S21, the biological development supply pile assembly is separated from a culture space assembly being completed cultivation of organisms on each layer of the preset bracket;
S22, organisms in an upper part first-layer culture space assembly to be harvested in each of the movable laminated biological culture devices on the preset bracket are harvested, then a harvested upper part first-layer culture space assembly pushed and transferred to a preset device by means of the slide rail part and the sliding part on each of the slideway walls;
S23, organisms are continuously harvested in an upper part second-layer culture space assembly to be harvested of each of the movable laminated biological culture devices, and then a harvested upper part second-layer culture space assembly is continuously pushed and transferred to a preset device by means of the slide rail part and sliding part on each of the slideway walls;
S24, organisms are continuously harvested in an upper part third-layer culture space assembly to be harvested in each of the movable laminated biological culture devices, and then a harvested upper part third-layer culture space assembly is continuously pushed and transferred to a preset device by means of the slide rail part and sliding part on each of the slideway walls;
S25, by analogy, until harvesting of organisms in each layer of culture space assembly is completed.

The embodiment of the disclosure also provides a using method for movable laminated biological culture devices, which includes movable laminated biological culture devices, applying the movable laminated biological culture devices to a joint operation of harvesting organisms to sowing biological seeds at least includes:
S1, the biological development supply pile assembly is separated from a culture space assembly of each layer having been completed cultivation of organisms;
S2, organisms in an upper part first-layer culture space assembly to be harvested in each of the movable laminated biological culture devices is harvested, then a bottom part of a first-layer culture space assembly having completed harvesting organisms is purified and biological seeds are sowed, and a seeded culture space assembly is transferred to a preset bracket to become a lower part first-layerculture space assembly, being about to enter the biological culture process again, of the preset bracket;
S3, organisms in an upper part second-layer culture space assembly to be harvested in each of the movable laminated biological culture devices are harvested, then a bottom part of a second-layer culture space assembly having completed harvesting organisms is purified and biological seeds are sowed, and a seeded culture space assembly is transferred to an upper part of a lower part first-layer culture space assembly, being about to enter the biological culture process again, of the preset bracket, so as to become the lower part second-layer culture space assembly, being about to enter the biological culture process again, of the preset bracket;
S4, organisms in an upper part third-layer culture space assembly to be harvested in each of the movable laminated biological culture devices are harvested, then a bottom part of a third-layer culture space assembly having completed harvesting organisms is purified and sowing biological seeds, and a seeded culture space assembly is transferred to an upper part of a lower part second-layer culture space assembly, being about to enter the biological culture process again, of the preset bracket, so as to become a lower part third-layer culture space assembly, being about to enter the biological culture process again, of the preset bracket;
S5, by analogy, harvesting organisms, purification treatment and sowing biological seeds are simultaneously completed for each layer of culture space assembly through joint operation, and the biological development supply pile assembly is connected with a culture space assembly having been seeded in each layer on the preset bracket, so as to continue cultivation of organisms until the cultivation of organisms is completed, and then a joint operation process of harvesting organisms, synchronous purification treatment and sowing is re-circulated.

The embodiment of the disclosure also provides a using method for a movable laminated biological culture device, which includes movable laminated biological culture devices, applying the movable laminated biological culture devices to product mushroom at least includes:
S1, the biological development supply pile assembly in each of the movable laminated biological culture devices is separated from each layer of culture space assembly;
S2, a bottom part and supporting walls in an upper part first-layer culture space assembly in each of the movable laminated biological culture devices are separated, mushroom culture substrate is simultaneously arranged on an upper part first-layer bottom part, and transferred a bottom part bearing the mushroom culture substrate to a preset bracket to become a lower part first-layer bottom part, being about to enter a substrate disinfection process, of the preset bracket;
S3, a bottom part and supporting walls in an upper part second-layer culture space assembly in each of the movable laminated biological culture devices are separated, and the mushroom culture substrate is simultaneously arranged on an upper part second-layer bottom part, and transferred a bottom part bearing the mushroom culture substrate to a lower part first-layer bottom part of the preset bracket to become a lower part second-layer bottom part, being about to enter the substrate disinfection process, of the preset bracket;
S4, a bottom part and supporting walls in an upper part third-layer culture space assembly in each of the movable laminated biological culture devices are separated, and the mushroom culture substrate is simultaneously arranged on an upper part third-layer bottom part, and transferred a bottom part bearing the mushroom culture substrate to a lower part second-layer bottom part on the preset bracket to become a lower part third-layer bottom part, being about to enter the substrate disinfection process, of the preset bracket;
S5, by analogy, a bottom part and supporting walls in each layer of culture space assembly in each of the movable laminated biological culture devices are separated, and arrangement of mushroom culture substrate on each layer of a bottom part is completed, and a bottom part, bearing the mushroom culture substrate, of each layer on a preset bracket is overlapped;
S6, a bottom part of each layer on the preset bracket together with bore mushroom culture substrate is sterilized;
S7, mushroom inoculation is performed on mushroom culture substrate on an upper part first-layer bottom part having been disinfected on the preset bracket, a bottom part having been completed mushroom inoculation is transferred to a new preset bracket to become a lower part first-layer bottom part of the new preset bracket, and one of the supporting walls is simultaneously arranged on the lower part first-layer bottom part to form lower part first-layer culture space of the new preset bracket, and the biological development factor supply part is arranged in the lower part first-layer culture space together with the supporting wall;
S8, mushroom inoculation is performed on an upper part second-layer bottom part having been disinfected on the preset bracket, a bottom part having been inoculated with mushroom is transferred to a supporting wall on the lower part first-layer bottom part of the new preset bracket, and another supporting wall is simultaneously arranged on the lower part first-layer bottom part to form a lower part second-layer culture space of the new preset bracket, and the biological development factor supply part is arranged in the lower part second-layer culture space together with the supporting wall;
S9, mushroom inoculation is performed on an upper part third-layer bottom part having been disinfected on the preset bracket, a bottom part having been inoculated with mushroom is transferred to a supporting wall on a lower part second-layer bottom part of the new preset bracket, and another supporting wall is simultaneously arranged on the lower part second-layer bottom part to form a lower part third-layer culture space of the new preset bracket, and the biological development factor supply part is arranged in the lower part third-layer culture space together with the supporting wall;
S10, by analogy, until inoculation of mushroom culture substrate on a bottom part of each layer is completed, and laminated culture space is re-formed, and then the biological development supply pile assembly is connected with a culture space assembly of each layer, and mushroom culture process is entered until mushroom culture is successful, and mushroom harvesting process is entered;
S11, the biological development supply pile assembly is separated from each layer of culture space assembly;
S12, mushroom in each culture space at an upper part of the new preset bracket is harvested, and supporting walls and a bottom part of each layer are separated for later use.

The technical scheme provided by each embodiment of the disclosure will be described in detail below with reference to the attached drawings.

### Embodiment 1

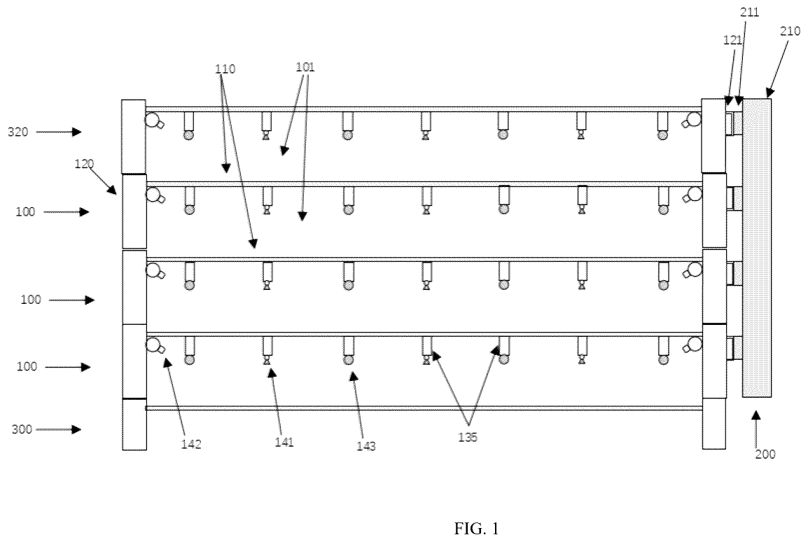

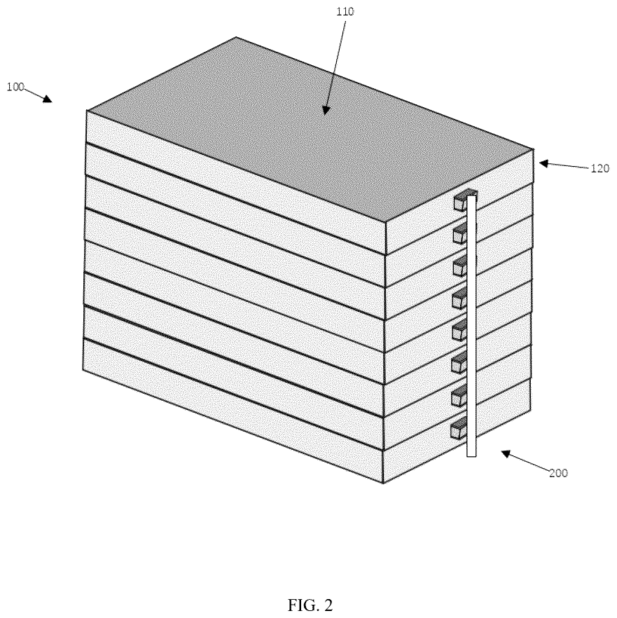

As shown in FIG. 1 and FIG. 2, a movable laminated biological culture device of Embodiment 1 includes a plurality of culture space assemblies 100 laminated up and down, a biological development factor supply part 140 coordinated with each of the culture space assemblies 100 being located in each layer, and a biological development supply pile assembly 200 coordinated with the biological development factor supply part 140. Each of the culture space assemblies 100 includes a bottom structure 110 and supporting walls 120, the upper end of the supporting wall 120 is connected to the bottom structure 110 at the edge of the bottom structure 110, and the bottom structure 110 and the last supporting wall 120 enclose with the last bottom structure 110 to form the culture space 101. The culture space 101 is used for biological culture, and the plurality of culture space assemblies 100 laminated up and down are connected with each other through respective the supporting walls 120. The uppermost culture space assembly 100 can be used as the top cover assembly 320 to close the next layer of culture space assembly 100. Where, that bottom structure of the bottom structure 110 is provide with a plurality of reinforcing structure 135 to slow down the downward depression of the bottom structure 110 under pressure when culturing organisms. The reinforcing structure 135 can be a bending bar, and its two ends are connected to the opposite supporting walls 120, so as to conduct the pressure to the supporting walls 120 connected with it.

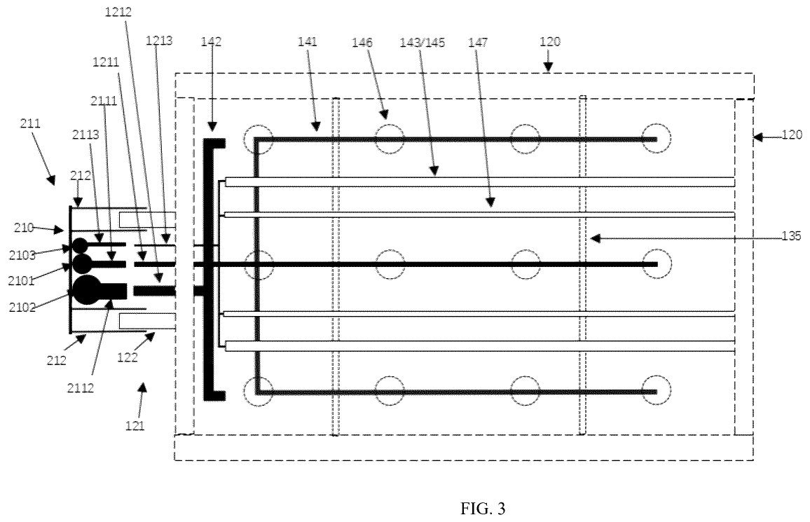

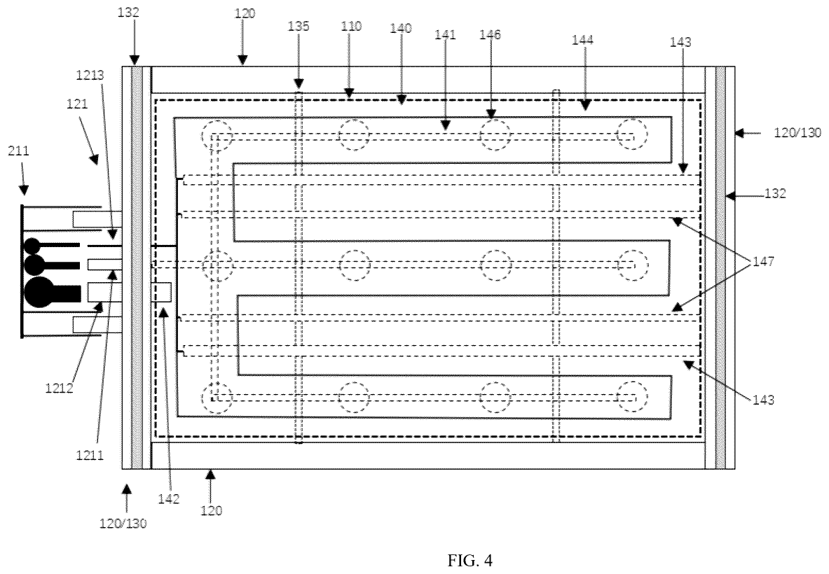

As shown in FIGS. 3 and 4, the biological development factor supply part 140 is arranged correspondingly to the culture space 101, and the biological development factor supply part 140 includes a supporting wall clutch part 121, a water body dispersion structure 141, a gas dispersion structure 142, a light emitter 143 and a heater 144. Where, the heater 144 is an electric heating device, which can share the power supply with the light emitter 143. There is no restriction on the arrangement of functional equipment such as the water dispersion structure 141, the gas dispersion structure 142, the light emitter 143 and the heater 144 in the culture space assembly 100. The supporting wall clutch part 121 is correspondingly provided with a water body receiving port 1211, a gas receiving port 1212 and a power receiving port 1213, correspondingly, the water body dispersion structure 141 is connected with the water body receiving port 1211, the gas dispersion structure 142 is connected with the gas receiving port 1212, and the light emitter 143 and the heater 144 are connected with the power receiving port 1213. The biological development factor supply part 140 are uniformly arranged on the bottom structure 110 and connected to the bottom structure 110 and the supporting wall 120 at one side. In addition, the biological development factor supply part 140 may further include a lamp holder 145, a sprayer 146 and a sterilization lamp 147.

Correspondingly, as shown in FIGS. 3 and 4, the biological development supply pile assembly 200 includes a pile frame 210 and a plurality of pile frame clutch parts 211 arranged on the pile frame 210, and the pile frame clutch parts 211 are arranged at intervals up and down corresponding to the biological development factor supply part 140. The pile frame clutch part 211 is provided corresponding to the supporting wall clutch part 121 and includes a water body supply port 2111, a gas supply port 2112 and a power supply port 2113, and the pile frame clutch part 211 is detachably connected with the corresponding supporting wall clutch part 121, In order to increase the stability of the connection between the pile frame clutch part 211 and the supporting wall clutch part 121, a pile frame clutch part stabilizer 212 and a supporting wall clutch part coupler 122 which can be matched for use are provided, and the pile frame clutch part stabilizer 212 can be clamped on the supporting wall clutch part coupler 122. The biological development supply pile assembly 200 further includes a water body supply part 2101, a gas supply part 2102 and a power supply part 2103 which are arranged corresponding to the pile frame clutch part 211. Where the water body supply part 2101 of the biological development supply pile assembly 200 is connected with the water body supply port 2111, the gas supply part 2102 is connected to the gas supply port 2112, the power supply part 2103 is connected to the power supply port 2113. The water body supply part 2101 is connected to the preset water source 2. The water body supply part 2101 is detachably connected with the water body receiving port 1211 of the supporting wall clutch part 121 through the water body supply port 2111 of the pile frame clutch part 211, and is used for supplying or cutting off the moisture necessary for the growth and development of organisms in the culture space 101 through the water dispersion structure 141.

Where, the gas supply part 2102 is connected to the preset gas source 3, and is detachably connected with the gas receiving port 1212 of the supporting wall clutch part 121 through the gas supply port 2112 of the pile frame clutch part 211, so as to supply or cut off the gas necessary for the growth and development of organisms in the culture space 101 through the gas dispersion structure 142. The power supply part 2103 is connected to the preset power supply 4. The power supply part 2103 is detachably connected to the power receiving port 1213 of the supporting wall clutch part 121 through the power supply port 2113 of the pile frame clutch part 211, and is used for supplying or cutting off the light and/or temperature necessary for the growth and development of organisms in the culture space 101 through the light emitter 143 and/or the heater 144.

At this time, the connection or separation mode of the water body supply port 2111 of the pile frame clutch part 211 and the water body receiving port 1211 of the supporting wall clutch part 121 is not limited in the disclosure, and can be realized by, for example, quick joint parts. The connection or separation mode of the gas supply port 2112 of the pile frame clutch 211 and the gas receiving port 1212 of the supporting wall clutch part 121 is not limit in the disclosure, for example, the pile frame 210 can be made of steel pipe, and the body cavity of the pile frame 210 can be directly butted or separate from the through hole reserved on the supporting wall 120. The connection or separation mode of the pow supply port 2113 of the pile frame clutch part 211 and the power receiving port 1213 of the supporting wall clutch part 121 is not limit in the disclosure, and can be realized by sockets and plugs, for example.

Further, as shown in FIG. 1, the supporting wall 120 is fixedly connected to the bottom structure 110, the biological development factor supply part 140 is fixedly connected to the culture space assembly 100, the supporting wall clutch part 121 is fixedly connected to the supporting wall 120, and the water dispersion structure 141, the gas dispersion structure 142, the light emitter 143 and the heater 144 are fixedly connected to the supporting wall 120 and/or the bottom structure 110, and the pile frame clutch part 211 is detachably connected with the culture space assembly 100 through the supporting wall clutch part 121. Where, in order to make full use of the culture space assemblyies 100 located at the uppermost layer and the lowermost layer, the uppermost culture space assembly 100 actually serves as the top cover assembly 320, and the bottom supporting assembly 300 at the lowermost layer functions as the bottom structure 110.

Optionally, as shown in FIGS. 1 and 2, the top ends of the supporting walls 120 are connected to the bottom structure 110 around, and the culture space 101 is formed by enclosing the bottom structure 110, the supporting walls 120 matched with the bottom structure 110 and the next bottom structure 110 below the bottom structure 110. After the biological development supply pile assembly 200 is connected with the supporting wall 120, at least one of moisture, air and power is supplied to the culture space 101.

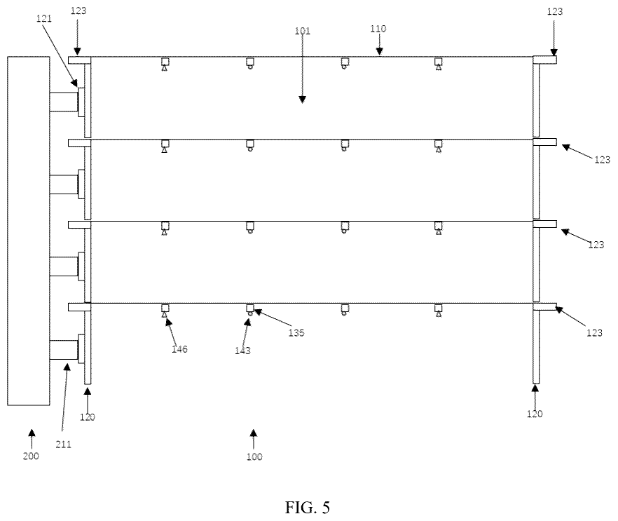

At the same time, as shown in FIG. 5, in order to facilitate the movement of each culture space assembly 100, a relocation structure 123 is fixedly connected to the upper part of each supporting wall 120. At this time, the relocation structure 123 serves as a gripper and can be used as a focus point when moving the culture space assembly 100.

When the movable laminated biological culture device in Embodiment 1 is used in a building, the building includes a beam-column structure 6, on which a supply pile connection part 310 is provided, the movable laminated biological culture device 1 is used in the building, and the biological development supply pile assembly 200 is arranged close to the supply pile connection part 310. The biological development supply pile assembly 200 is connected or separated from the beam-column structure 6 through the supply pile connection part 310. Before the culture space assembly 100 is activated, the biological development supply pile assembly 200 is connected with the beam-column structure 6 through the supply pile connection part 310, and at least one of the preset water source 2, preset gas source 3 and preset power source 4 is connected with the beam-column structure 6 and connected with the biological development supply pile assembly 200 along the beam-column structure 6. The using method at least includes the following steps:

When it is necessary to supply at least one of moisture, gas and power to the culture space 101 of the movable laminated biological culture device 1, the biological development supply pile assembly 200 is separated from the beam-column structure 6, and the pile frame clutch part 211 of the biological development supply pile assembly 200 is connected with the culture space assembly 100 through the supporting wall clutch part 121.

When the culture space assembly 100 of the movable laminated biological culture device 1 needs to be transported and moved, the pile frame clutch part 211 of the biological development supply pile assembly 200 is separated from the supporting wall clutch part 121, so that the biological development supply pile assembly 200 is separated from the culture space assembly 100, and then the biological development supply pile assembly 200 is connected with the beam-column structure 6 through the supply pile connection part 310.

### Embodiment 2

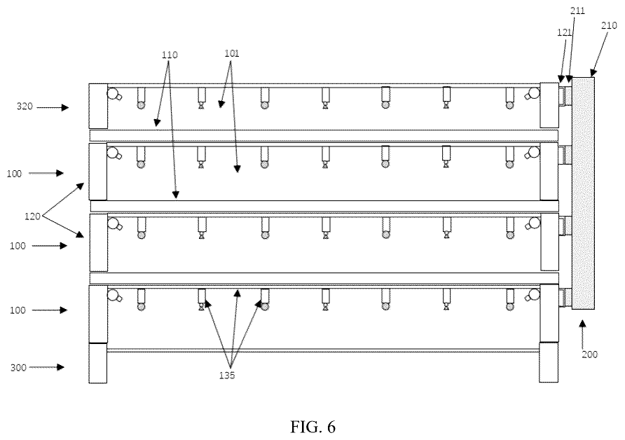

As shown in FIG. 6, the movable laminated biological culture device of Embodiment 2 is different from Embodiment 1 in that the supporting wall 120 is detachably connected to the bottom structure 110, and the biological development factor supply part 140 is detachably connected to the culture space assembly 100. The supporting wall clutch part 121, the water body dispersion structure 141, the gas dispersion structure 142, the light emitter 143 and the heater 144 are all fixedly connected to the supporting wall 120, and the biological development factor supply part 140 is connected with the culture space assembly 100 along with the connection of the supporting wall 120 and the bottom structure 110. With the separation of the supporting wall 120 and the bottom structure 110, the biological development factor supply part 140 is actually separated from the culture space assembly 100, and the pile frame clutch part 211 is detachably connected with the culture space assembly 100 through the supporting wall clutch part.

The movable laminated biological culture device of Embodiment 2 is suitable for mushroom culture. During mushroom culture, it is necessary to sterilize the mushroom culture substrate 7 on the bottom structure 110 at high temperature, which can separate the bottom structure 110 from the supporting wall 120. Accordingly, the biological development factor supply part 140 is separated from the bottom structure 110 in the process of high temperature disinfection along with the supporting wall 120, so as to avoid the biological development factor supply part 140 from being damaged by high temperature.

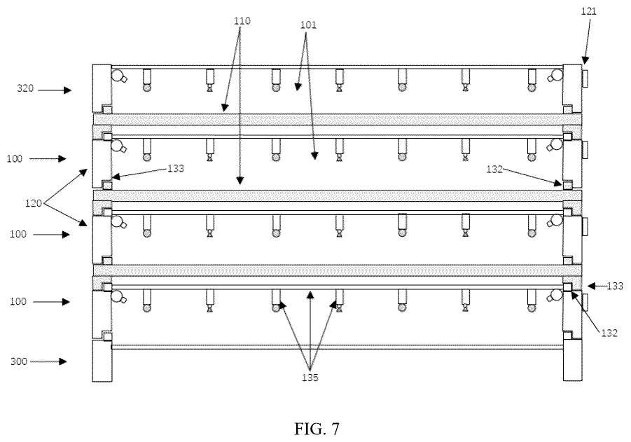

Optionally, as shown in FIG. 7, the bottom structure 110 is rectangular, and the supporting wall 120 includes two slideway walls 130 which are arranged oppositely, and the top part and bottom structure of the slideway wall 130 are respectively provided with a slide rail structure 132 and a sliding structure 133 which can be mutually used up and down. The upper and lower parts of the bottom structure 110 corresponding to the slideway wall 130 are respectively provided with a slide rail structure 132 and a sliding structure 133 which can be can be used in cooperation with the slideway wall 130 up and down, and the bottom structure 110 can slide away from the supporting wall 120 located below along the slide rail structure 132 or the sliding structure 133 on the slideway wall 130. The supporting wall 120 can slide away from the bottom structure 110 located below along the sliding rail structure 132 or sliding structure 133 on the bottom structure 110, and the biological development supply pile assembly 200 is connected with the supporting wall 120 on at least one of the four sides of the bottom structure 110.

### Embodiment 3

The movable laminated biological culture device of Embodiment 3 is different from Embodiment 1 in that the middle parts of the supporting walls 120 are connected to the bottom structure 110 around. The culture space 101 is formed by enclosing a bottom structure 110, supporting walls 120 matched with the bottom structure 110, a previous bottom structure 110 above the bottom structure 110 and a previous supporting wall 120 above the supporting wall 120. After being connected with the supporting wall 120 or the previous supporting wall 120 above the supporting wall 120, the biological development supply pile assembly 200 supplies at least one of moisture, air and electricity to the culture space 101.

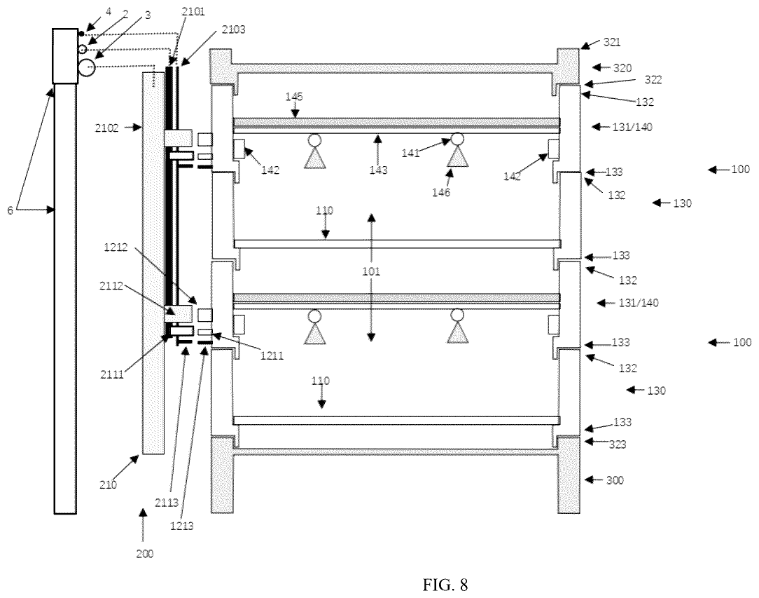

Optionally, as shown in FIG. 8, the supporting wall 120 adopts a clutch supporting wall 131, which includes a supporting wall fixedly connected with the biological development factor supply part 140 and a supporting wall fixedly connected with the bottom structure 110. The supporting wall fixedly connected with the biological development factor supply part 140 and the supporting wall fixedly connected with the bottom structure 110 are respectively provided with a sliding rail structure 132 and a sliding structure 133 which can slide in cooperation with each other. In the process of using the movable laminated biological culture device 1, the biological development supply pile assembly 200 is alternately connected and separated from the beam-column structure 6 and the culture space assembly 100. Meanwhile, the top of the top cover assembly is provided with a disk cover slide rail structure 321 and the bottom is provided with a disk cover sliding structure 322, which can be slidably connected with the slide rail structure 132 on the top of the supporting wall 120, and the top of the bottom supporting assembly 300 is provided with a bracket slide rail structure 323, which can be slidably connected with the sliding structure 133 on the bottom of the supporting wall 120.

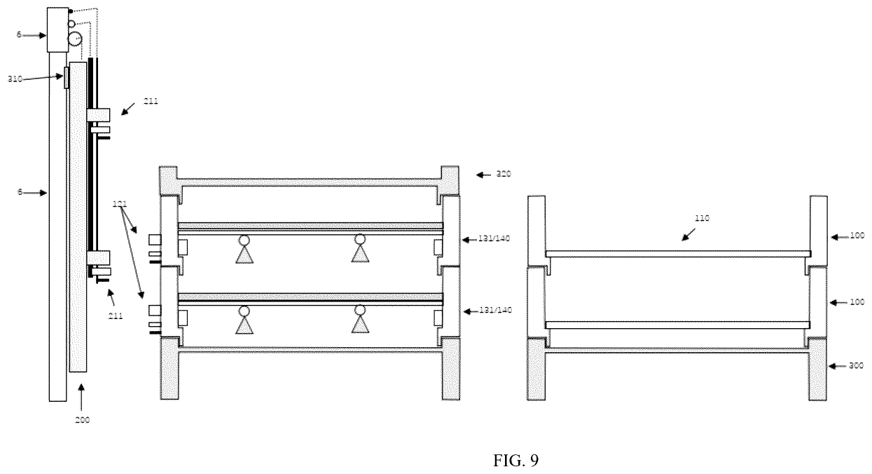

On the one hand, as shown in FIG. 9, when working on the bottom structure 110, the supporting wall 120 connected with the biological development factor supply part 140 can be temporarily separated from the bottom structure 110 to facilitate the work.

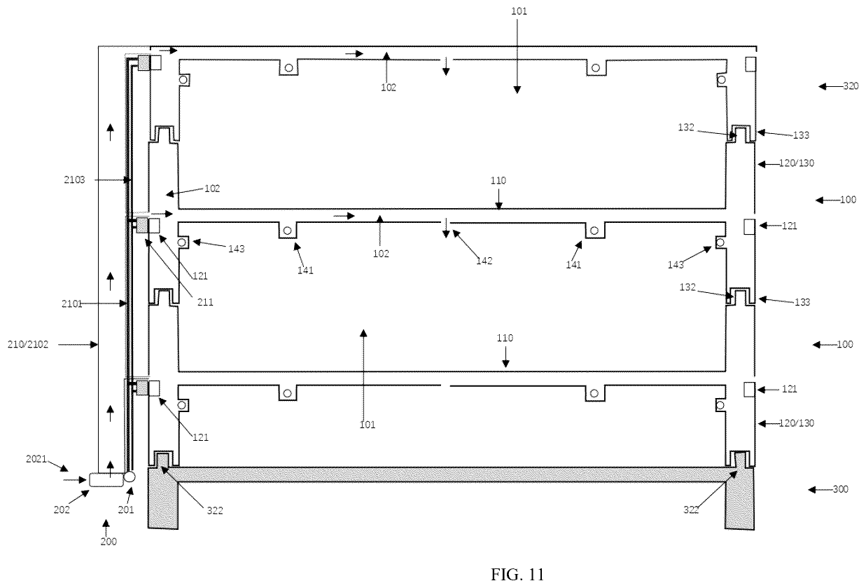

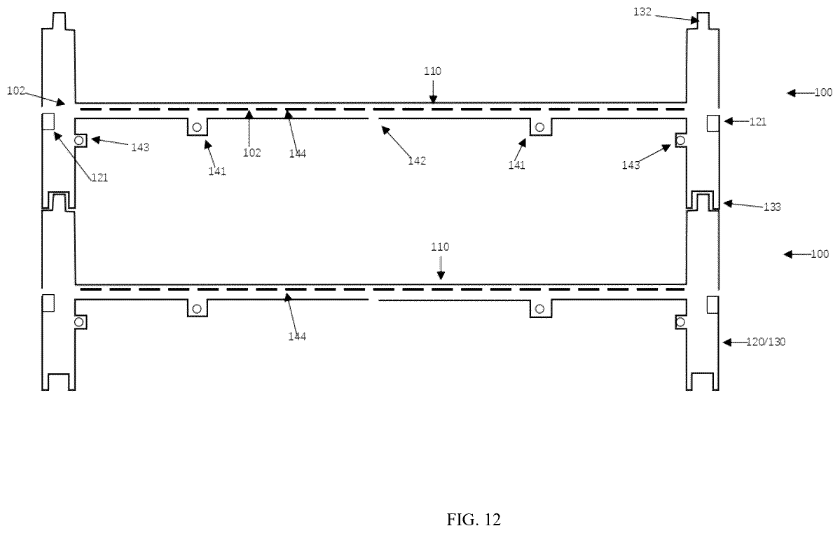

On the other hand, as shown in FIG. 11, the supporting wall 120 and the bottom structure 110 can be designed as an integrated structure, and both the supporting wall 120 and the bottom structure 110 are provided with communicated air flow cavities 102, and the water body dispersion structure 141, the gas dispersion structure 142 and the light emitter 143 in the biological development factor supply part 140 are all connected to the integrated structure. The airflow cavity 102 can be used as a gas dispersion structure 142. The biological development supply pile assembly 200 is also provided with a water flow booster pump 201 and an air flow pump 202, and the air flow 2021 enters the gas supply part 2102 through the air flow pump 202. This embodiment is suitable for using steel pipes as the skeleton structure of the supporting wall 120 and the skeleton structure of the bottom structure 110. After the pipe cavities of the steel pipes are communicated, they are used to install the water body dispersion structure 141, the gas dispersion structure 142 and the light emitter 143 in the biological development factor supply part 140. Alternatively, in this embodiment, an injection-molded part made by injection molding is also suitable as the skeleton structure of the supporting wall 120 and the skeleton structure of the bottom structure 110, and after the cavities of the injection-molded part are communicated, it is used to install the water body dispersion structure 141, the gas dispersion structure 142 and the light emitter 143 in the biological development factor supply part 140, and as shown in FIG. 12, a heater 144 may also be provided. The heater 144 is an electric hot wire, which is arranged in the bottom structure 110. The heater 144 can be embedded in the injection molding part when the bottom structure 110 is manufactured by injection molding.

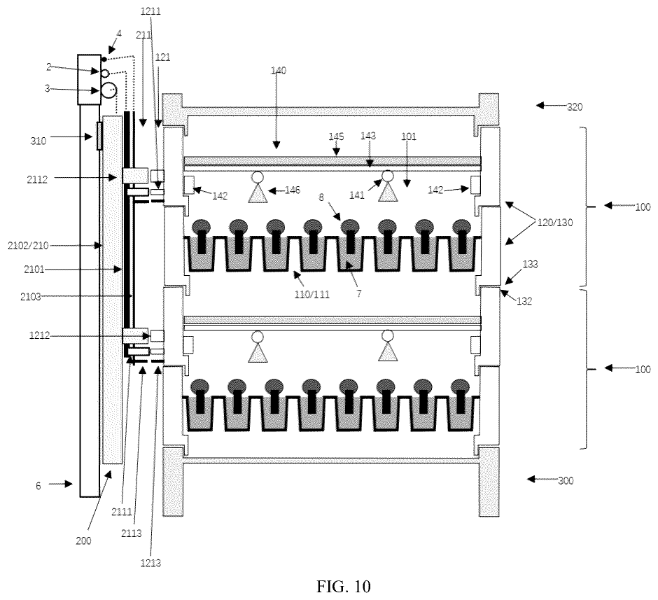

Optionally, as shown in FIG. 10, the bottom structure 110 includes a plurality of pits 111 arranged downwards, and the pits 111 are used for bearing nutrient substrates needed for cultivating preset organisms, so that the preset organisms can absorb nutrients from the nutrient substrates in the pits 111, and the biological development supply pile assembly 200 is used for supplying at least one of moisture and air to the nutrient substrates in the pits 111. At this time, the movable laminated biological cultivation device of Embodiment 3 is suitable for cultivating mushroom 8, and it is also suitable for cultivating vegetables such as lettuce.

### Embodiment 4

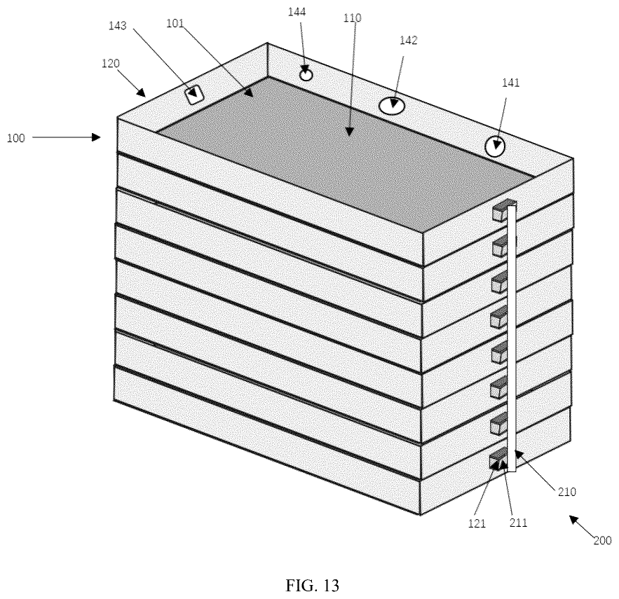

As shown in FIG. 13, the movable laminated biological culture device of Embodiment 4 is different from Embodiment 1 in that the bottom end of the supporting wall 120 is connected to the bottom structure 110 around, and the culture space 101 is formed by enclosing the bottom structure 110, the supporting wall 120 matched with the bottom structure 110 and the previous bottom structure 110 located above the bottom structure 110. After the biological development supply pile assembly 200 is connected with the supporting wall 120, moisture, air and water are supplied to the culture space 101.

Where, the water body dispersion structure 141, the gas dispersion structure 142, the light emitter 143 and the heater 144 of the biological development factor supply part 140 are all arranged on the supporting wall 120, which is suitable for insect larva culture.

### Embodiment 5

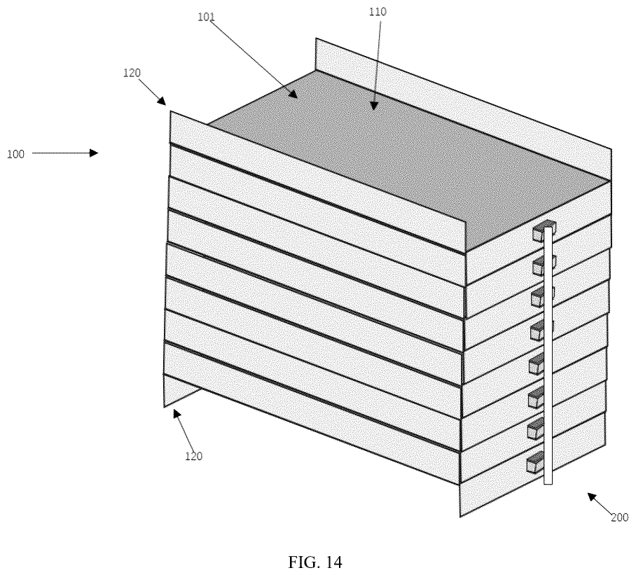

As shown in FIG. 14, the movable laminated biological culture device of Embodiment 5 is different from Embodiment 1 in that the bottom structure 110 is rectangular, and four supporting walls 120 include two oppositely arranged supporting walls 120 extending upward along the bottom structure 110 and two oppositely arranged supporting walls 120 extending downward along the bottom structure 110. The culture space 101 is formed by enclosing a bottom structure 110, two supporting walls 120 extending upward along the bottom structure 110, a previous bottom structure 110 located above the bottom structure 110 and two supporting walls 120 extending downward from the previous bottom structure 110. The biological development supply pile assembly 200 is connected with the supporting wall 120 extending upward along the bottom structure 110 or the supporting wall 120 extending downward along the bottom structure 110.

The movable laminated biological culture device of Embodiment 5 is suitable for setting a slide rail structure 132 and a slide structure 133 on two supporting walls 120 extending upward along the bottom structure 110, and then becoming a slideway wall 130, and it is also suitable to provide a supporting wall 120 extending downward along the bottom structure 110 as a harvesting wall 134.

### Embodiment 6

As shown in FIG. 11, the movable laminated biological culture device of Embodiment 6 is different from Embodiment 1 in that the bottom structure 110 is rectangular, and the supporting walls 120 include two oppositely arranged slideway wall 130, and the top and bottom of the slideway wall 130 are respectively provided with a slide rail structure 132 and a sliding structure 133 which can be cooperated with each other up and down. The culture space assembly 100 located on on the previous layer can slide away from the culture space assembly 100 located on the next layer along the sliding rail structure 132 or sliding structure 133 on the slideway wall 130, and the biological development supply pile module 200 is connected with the supporting wall 120 on at least one of the four sides of the bottom structure 110.

Preferably, as shown in FIG. 12, the other two supporting walls 120 vertically opposite to the two slideway walls 130 are harvesting walls 134, and the length of the harvesting walls 134 extending downward along the bottom structure 110 is less than that of the slideway walls 130 extending downward along the bottom structure 110. So that the culture space assembly 100 on the previous layer slides away from the culture space module 100 on the next layer along the slide rail structure 132 or the slide structure 133 on the slideway wall 130, and at the same time, the preset position of the organism cultured in the culture space assembly 100 on the next layer is harvested and pushed away in the same direction by means of a harvesting wall 134 of the culture space assembly 100 on the previous layer, the harvesting of organisms is realized, the biological development supply pile assembly 200 is connected with at least one supporting wall 120 in the slideway wall 130 and/or the harvesting wall 134.

Where, the sliding structure 133 arranged on the slideway wall 130 can be a roller structure, and correspondingly, the slide rail structure 132 can be a groove structure or a convex structure.

### Embodiment 7

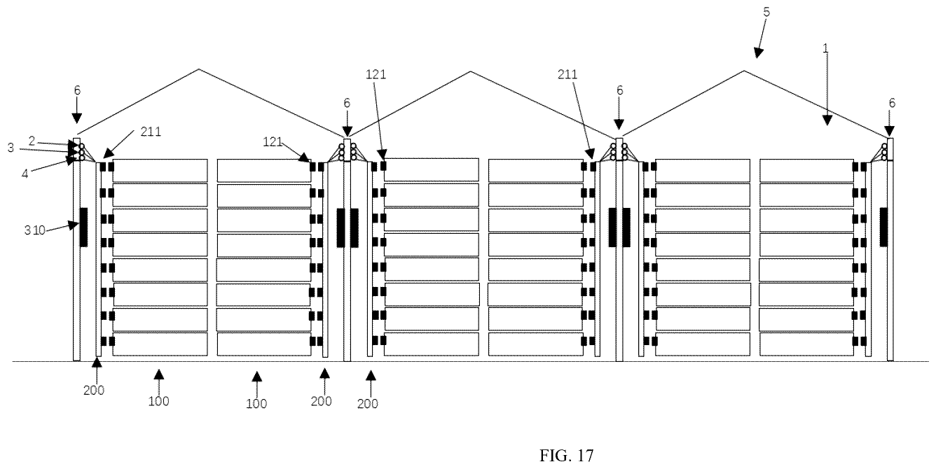

As shown in FIG. 17, the using method of the movable laminated biological culture device in Embodiment 7 includes the movable laminated biological culture devices 1 and buildings, the building includes a beam-column structure 6, and the beam-column structure 6 is provided with a supply pile connection part 310, and the movable laminated biological culture device 1 is arranged in the building for use. The biological development supply pile assembly 200 is arranged close to the supply pile connection part 310, and the biological development supply pile assembly 200 is connected or separated with the beam-column structure 6 through the supply pile connection part 310. Before the culture space assembly 100 is activated, the biological development supply pile assembly 200 is connected with the beam-column structure 6 through the supply pile connection part 310. At this time, at least one of the preset water source 2, preset gas source 3 and preset power source 4 is connected to the beam-column structure 6 and connected to the biological development supply pile assembly 200 along the beam-column structure 6. The using method at least includes the following steps:

When it is necessary to supply at least one of moisture, gas and power to the culture space 101 of the movable laminated biological culture device 1, the biological development supply pile assembly 200 is separated from the beam-column structure 6, and the pile frame clutch part 211 of the biological development supply pile assembly 200 is connected with the culture space assembly 100 through the supporting wall clutch part 121.

When the culture space assembly 100 of the movable laminated biological culture device 1 needs to be transported and moved, the pile frame clutch part 211 of the biological development supply pile assembly 200 is separated from the supporting wall clutch part 121, so that the biological development supply pile assembly 200 is separated from the culture space assembly 100, and then the biological development supply pile assembly 200 is connected with the beam-column structure 6 through the supply pile connection part 310.

At this time, the biological development supply pile assembly 200 is close to the beam-column structure 6 and is arranged at one side of the culture space assembly 100, so that the biological development supply pile assembly 200 is alternately connected and separated from the beam-column structure 6 and the culture space assembly 100 during the use of the movable laminated biological culture device 1, and the building in this embodiment at least includes the multi-span greenhouse 5.

### Embodiment 8

The use method of the movable laminated biological culture device in Embodiment 8 includes the movable laminated biological culture device 1 in any one of Embodiments 1-5, and the movable laminated biological culture device 1 is used for the cultivation of organisms, and a process from sowing biological seeds to harvesting organisms at least includes:
S1, biological culture process
S11, biological seeds are sowed to a bottom structure 110 of an upper part first-layer culture space assembly 100 to be seeded in the movable laminated biological culture device 1, and then a seeded culture space assembly 100 is suspended upward to be separated from the upper part second-layer culture space assembly 100 and then transferring to a preset bracket, so as to become a lower part first-layer culture space assembly 100, being about to enter the biological culture process, of the preset bracket;
S12, biological seeds are continuously sowed to a bottom structure 110 of an upper part second-layer culture space assembly 100 to be seeded in the movable laminated biological culture device 1, and then a seeded culture space assembly 100 is suspended upward to be separated from an upper part third-layer culture space assembly 100, and then transferred to above a lower part first-layer culture space assembly 100, being about to enter the biological culture process. of the preset bracket, so as to become a lower part second-layer culture space assembly 100, being about to enter the biological culture process, of the preset bracket;
biological seeds are continuously sowed to a bottom structure 110 of an upper part third-layer culture space assembly 100 to be seeded in the movable laminated biological culture device 1, then a seeded culture space assembly 100 is suspended upward to be separated from an upper part fourth-layer culture space assembly 100, and then transferred to above a culture space assembly 100 being about to enter the biological culture process in a lower part second-layer of the preset bracket, so as to become a culture space assembly 100 being about to enter the biological culture process in a lower part third-layer of the preset bracket;
S14, by analogy, until sowing biological seeds of each layer of culture space assembly 100 is completed, and the biological development supply pile assembly 200 is connected with a seeded culture space assembly 100 in each layer on the preset bracket, so as to perform cultivation of organisms, until the cultivation of organisms is completed, and entering harvesting organisms process;
S2, harvesting organisms process
S21, the biological development supply pile assembly 200 is separated from a culture space assembly 100 being completed cultivation of organisms on each layer of the preset bracket;
S22, organisms in an upper part first-layer culture space assembly 100 to be harvested in the movable laminated biological culture device 1 on the preset bracket are harvested, and then a harvested culture space assembly 100 is suspended upward to be separated from the upper part second-layer culture space assembly and transferred to a preset device;
S23, organisms in an upper part second-layer culture space assembly 100 to be harvested of the movable laminated biological culture device 1 are continuously harvested, and then a harvested culture space assembly 100 is suspended upward to be separated from the upper part third-layer of the culture space assembly 100, and then transferred to a preset device;
S24, organisms in an upper part third-layer culture space assembly 100 to be harvested in the movable laminated biological culture device 1 are continuously harvested, and then a harvested culture space assembly 100 is suspended upward to be separated from an upper part fourth-layer culture space assembly 100 and transferred to a preset device;
S25, by analogy, until harvesting of organisms in each layer of culture space assembly 100 is completed.

### Embodiment 9

The using method of the movable laminated biological culture device of Embodiment 9 includes the movable laminated biological culture device 1 of Embodiment 6, and the movable laminated biological culture device 1 is used for biological culture, and a process from sowing biological seeds to harvesting organisms at least includes:
S1, biological culture process
S11, biological seeds are sowed to a bottom structure 110 of an upper part first-layer culture space assembly 100 to be seeded in the movable laminated biological culture device 1, and then an upper part first-layer culture space assembly 100 having been seeded is pushed and transferred to the preset bracket by means of the slide rail structure 132 and the sliding structure 133 on the slideway wall 130, to become a culture space assembly 100 being about to enter the biological culture process in a lower part first-layer of the preset bracket;
S12, biological seeds are continuously sowed to a bottom structure 110 of an upper part second-layer culture space assembly 100 to be seeded in the movable laminated biological culture device 1, and then an upper part second-layer culture space assembly 100 having been seeded is continuously pushed and transferred to above a lower part first-layer culture space assembly 100, being about to enter the biological culture process, of the preset bracket by means of the slide rail structure 132 and the sliding structure 133 on the slideway wall 130, so as to become a lower part second-layer culture space assembly 100, being about to enter the biological culture process, of the preset bracket;
S13, biological seeds are continuously sowed to a bottom structure 110 of an upper part third-layer culture space assembly 100 to be seeded in the movable laminated biological culture device 1, and then an upper part third-layer culture space assembly 100 having been seeded is continuously pushed and transferred to above a lower part second-layer culture space assembly 100, being about to enter the biological culture process, of the preset bracket by means of the slide rail structure 132 and sliding structure 133 on the slideway wall 130, so as to become a lower part third-layer culture space assembly 100, being about to enter the biological culture process, of the preset bracket;
S14, by analogy, until sowing biological seeds of each layer of culture space assembly 100 is completed, and the biological development supply pile assembly 200 is connected with a seeded culture space assembly 100 in each layer on the preset bracket, so as to perform cultivation of organisms until the cultivation of organisms is completed, and harvesting organisms process is entered;
S2, harvesting organisms process
S21, the biological development supply pile assembly 200 is separated from a culture space assembly 100 being completed cultivation of organisms on each layer of the preset bracket;
S22, organisms in an upper part first-layer culture space assembly 100 to be harvested in the movable laminated biological culture device 1 on the preset bracket are harvested, then a harvested upper part first-layer culture space assembly 100 is pushed and transferred to a preset device by means of the slide rail structure 132 and the sliding structure 133 on the slideway wall 130;
S23, organisms in an upper part second-layer culture space assembly 100 to be harvested of the movable laminated biological culture device 1 are continuously harvested, and then a harvested upper part second-layer culture space assembly 100 is continuously pushed and transferred to a preset device by means of the slide rail structure 132 and sliding structure 133 on the slideway wall 130;
S24, organisms in an upper part third-layer culture space assembly 100 to be harvested in the movable laminated biological culture device 1 are continuously harvested, and then a harvested upper part third-layer culture space assembly 100 is continuously pushed and transferred to a preset device by means of the slide rail structure 132 and sliding structure 133 on the slideway wall 130;
S25, by analogy, until harvesting of organisms in each layer of culture space assembly 100 is completed.

### Embodiment 10

The using method of the movable laminated biological culture device in Embodiment 10 includes the movable laminated biological culture device 1, and the movable laminated biological culture device 1 is used for the joint operation of harvesting organisms to sowing biological seeds, at least including the following steps:
S1, the biological development supply pile assembly 200 is separated from a culture space assembly 100 of each layer having been completed cultivation of organisms;
S2, organisms in an upper part first-layer culture space assembly 100 to be harvested in the movable laminated biological culture device 1 are harvested, then biological seeds are sowed to a bottom structure 110 of a first-layer culture space assembly 100 having completed harvesting organisms, and finally, a seeded culture space assembly 100 is transferred to a preset bracket to become a lower part first-layer culture space assembly 100, being about to enter the biological culture process again, of the preset bracket;
S3, organisms in an upper part second-layer culture space assembly 100 to be harvested in the movable laminated biological culture device 1 are harvested, then biological seeds are sowed to a bottom structure 110 of a second-layer culture space assembly 100 having completed harvesting organisms, and finally, a seeded culture space assembly 100 is transferred to an upper part of the lower part first-layer culture space assembly 100, being about to enter the biological culture process again, of the preset bracket, so as to become the lower part second-layer culture space assembly 100, being about to enter the biological culture process again, of the preset bracket;
S4, organisms in an upper part third-layer culture space assembly 100 to be harvested in the movable laminated biological culture device 1 are harvested, then biological seeds are sowed to a bottom structure 110 of a third-layer culture space assembly 100 having completed harvesting organisms, and finally, a seeded culture space assembly 100 is transferred to an upper part of a lower part second-layer culture space assembly 100, being about to enter the biological culture process again, of the preset bracket, so as to become a lower part third-layer culture space assembly 100, being about to enter the biological culture process again, of the preset bracket;
S5, by analogy, harvesting organisms, purification treatment and sowing biological seeds for each layer of culture space assembly 100 are simultaneously completed through joint operation, and the biological development supply pile assembly 200 is connected with a culture space assembly 100 having been seeded in each layer on the preset bracket, so as to continue cultivation of organisms until the cultivation of organisms is completed, and then a joint operation process of harvesting organisms, synchronous purification treatment and sowing is circularly entered.

### Embodiment 11

The using method of the movable laminated biological culture device in Embodiment 11 includes the movable laminated biological culture device 1, and the movable laminated biological culture device 1 is used for the production of mushroom 8, which at least includes the following steps:
S1, the biological development supply pile assembly 200 in the movable laminated biological culture device 1 is separated from each layer of culture space assembly 100;
S2, a bottom structure 110 and supporting walls 120 in an upper part first-layer culture space assembly 100 in the movable laminated biological culture device 1 are separated, mushroom culture substrate 7 is simultaneously arranged on an upper part first-layer bottom structure 110, and a bottom structure 110 bearing the mushroom culture substrate 7 is transferred to a preset bracket to become a lower part first-layer bottom structure 110, being about to enter a substrate disinfection process, of the preset bracket;
S3, a bottom structure 110 and supporting walls 120 in an upper part second-layer culture space assembly 100 in the movable laminated biological culture device 1 are separated, and the mushroom culture substrate 7 is simultaneously arranged on an upper part second-layer bottom structure 110, and a bottom structure 110 bearing the mushroom culture substrate 7 is transferred to a lower part first-layer bottom structure 110 of the preset bracket to become a bottom structure 110 of a lower part second-layer, being about to enter the substrate disinfection process, of the preset bracket;
S4, a bottom structure 110 and supporting walls 120 in an upper part third-layer culture space assembly 100 in the movable laminated biological culture device 1 are separated, and the mushroom culture substrate 7 is simultaneously arranged on an upper part third-layer bottom structure 110, and a bottom structure 110 bearing the mushroom culture substrate 7 is transferred to a lower part second-layer bottom structure 110 on the preset bracket to become a lower part third-layer bottom structure 110, being about to enter the substrate disinfection process, of the preset bracket;
S5, by analogy, a bottom structure 110 and supporting walls 120 in each layer of culture space assembly 100 in the movable laminated biological culture device 1 are separated, and arrangement of mushroom culture substrate 7 on each layer of a bottom structure 110 is completed, and a bottom structure 110 bearing the mushroom culture substrate 7, of each layer is overlapped on a preset bracket;
S6, a bottom structure 110 of each layer on the preset bracket together with bore mushroom culture substrate 7 is sterilized;
S7, mushroom inoculation is performed on mushroom culture substrate 7 on an upper part first-layer bottom structure 110 having been disinfected on the preset bracket, a bottom structure 110 having been completed mushroom inoculation is transferred to a new preset bracket to become a lower part first-layer bottom structure 110 of the new preset bracket, and one of the supporting walls 120 is simultaneously arranged on the lower part first-layer bottom structure 110 to form lower part first-layer culture space 101 of the new preset bracket, and the biological development factor supply part 140 is arranged in the lower part first-layer culture space 101 together with the supporting wall 120;
S8, mushroom inoculation is performed on mushroom culture substrate 7 in an upper part second-layer bottom structure 110 having been disinfected on the preset bracket, a bottom structure 110 having been inoculated with mushroom is transferred to a supporting wall 120 on the lower part first-layer bottom structure 110 of the new preset bracket, and another supporting wall 120 is simultaneously arranged on the lower part first-layer bottom structure 110 to form a lower part second-layer culture space 101 of the new preset bracket, and the biological development factor supply part 140 is arranged in the lower part second-layer culture space 101 together with the supporting wall 120;
S9, mushroom inoculation is performed on the mushroom culture substrate 7 in an upper part third-layer bottom structure 110 having been disinfected on the preset bracket, a bottom structure 110 having been inoculated with mushroom is transferred to a supporting wall 120 on a lower part second-layer bottom structure 110 of the new preset bracket, and another supporting wall 120 is simultaneously arranged on the lower part second-layer bottom structure 110 to form a lower part third-layer culture space 101 of the new preset bracket, and the biological development factor supply part 140 is arranged in the lower part third-layer culture space 101 together with the supporting wall 120;
S10, by analogy, until inoculation of mushroom culture substrate 7 on a bottom structure 110 of each layer is completed, and laminated culture space 101 is re-formed, and then the biological development supply pile assembly 200 is connected with a culture space assembly 100 of each layer, and mushroom culture process is entered until mushroom culture is successful, and mushroom harvesting process is entered;
S11, the biological development supply pile assembly 200 is separated from each layer of culture space assembly 100;
S12, mushroom in each culture space 101 at an upper part of the new preset bracket is harvested, and supporting walls 120 and a bottom structure 110 of each layer are separated for later use.

The movable laminated biological culture device and the using method thereof in the disclosure, in the culture process of industrialized plants, mushrooms, and insects, by arranging a plurality of biological development factor supply parts matched with each layer of culture space and a biological development supply pile assembly matched with each biological development factor supply part, a stack of multi-layer laminated culture space assemblies can be used together with another stack of multi-layer laminated culture space assemblies, and there is no need to set an operation channel, thus solving the problem that the reserved operation channel wastes land and space in the building facilities of the biological factory in the prior art.

At the same time, because the biological culture space of a movable laminated biological culture device according to the disclosure is movable, the biological seeds sowing operation, the biological culture operation and the biological harvesting operation can be flexibly realized by moving the biological culture space. Not only can each layer of a stack of multi-layer laminated culture space assemblies be separately transferred to a preset site to perform biological seeds sowing operation, biological culture operation and biological harvesting operation, but also a stack of multi-layer laminated culture space assemblies can be transferred to a preset site as a whole to perform biological seeds sowing operation, biological culture operation and biological harvesting operation on each layer of culture space respectively. Thereby the problems of great difficulty, low operating efficiency and high operating cost in seeding and harvesting cultivation sites in the prior art are solved. The disclosure has reasonable overall structural design and convenient use, and is suitable for popularization and application.

The above-mentioned embodiments only describe the preferred mode of the disclosure, and do not limit the scope of this disclosure. Under the premise of not departing from the design spirit of the disclosure, various modifications and improvements made by ordinary skilled in the field to the technical scheme of the disclosure shall fall within the scope of protection determined by the claims of the disclosure.

What is claimed is:

1. A movable laminated biological culture device, comprising a plurality of culture space assemblies laminated up and down, a biological development factor supply part coordinated with each of the culture space assemblies being located in each layer, and a biological development supply pile assembly coordinated with the biological development factor supply part, wherein each of the culture space assemblies comprises a bottom part and supporting walls, the supporting walls are connected to the bottom part at edges of the bottom part in a preset manner, and the bottom part and the supporting walls are enclosed in a preset manner to form a culture space, and the culture space is used for biological culture, and the plurality of culture space assemblies laminated up and down are detachably connected with each other through respective the supporting walls; the biological development factor supply part is arranged correspondingly to the culture space, and the biological development factor supply part comprises a supporting wall clutch part and further at least comprises one of a water body dispersion part, a gas dispersion part, a light emitter and a heater, wherein the supporting wall clutch part is correspondingly provided with at least one of a water body receiving port, a gas receiving port and a power receiving port, and the biological development factor supply part is connected with the supporting walls and/or the bottom part of the culture space in a preset manner; the biological development supply pile assembly comprises a pile frame and a plurality of pile frame clutch parts arranged on the pile frame at intervals from top to bottom, wherein each of the pile frame clutch parts is correspondingly arranged with the biological development factor supply part, and each of the pile frame clutch parts is also correspondingly arranged with the supporting wall clutch part and at least comprises one of a water body supply port, a gas supply port and a power supply port, and each of the pile frame clutch parts is detachably connected with corresponding supporting wall clutch part; the biological development supply pile assembly further comprises at least one of a water body supply part, a gas supply part and a power supply part arranged corresponding to each of the pile frame clutch parts, wherein the water body supply part is connected with a preset water source, and the water supply part is detachably connected with the water body receiving port of the supporting wall clutch part through the water body supply port of each of the pile frame clutch parts, so as to provide or cut off moisture necessary for growth and development of organisms in the culture space through the water body dispersion part; the gas supply part is connected with a preset gas source, and the gas supply part is detachably connected with the gas receiving port of the supporting wall clutch part through the gas supply port of each of the pile frame clutch parts, so as to provide or cut off gas necessary for growth and development of the organisms in the culture space through the gas dispersion part; the power supply part is connected to a preset power supply, and the power supply part is detachably connected with the power receiving port of the supporting wall clutch part through the power supply port of each of the pile frame clutch parts, so as to provide or cut off illumination and/or temperature necessary for growth and development of the organisms in the culture space through the light emitter and/or the heater.

2. The movable laminated biological culture device according to claim 1, wherein the supporting walls are fixedly connected to the bottom part, the biological development factor supply part is fixedly connected to each of the culture space assemblies, the supporting wall clutch part is fixedly connected to the supporting walls, and the water body dispersion part, the gas dispersion part, the light emitter and the heater are fixedly connected to corresponding one of the supporting wall and/or the bottom part, each of the pile frame clutch parts is detachably connected with each of the culture space assemblies through the supporting wall clutch part.

3. The movable laminated biological culture device according to claim 1, wherein the supporting walls are detachably connected to the bottom part, the biological development factor supply part is detachably connected to each of the culture space assemblies, and the supporting wall clutch part, the water body dispersion part, the gas dispersion part, the light emitter and the heater are all fixedly connected to the supporting walls; the biological development factor supply part is connected with each of the culture space assemblies along with connection of the supporting walls and the bottom part, the biological development factor supply part is separated from each of the culture space assemblies along with separation of the supporting walls and the bottom part, and each of the pile frame clutch parts is detachably connected with each of the culture space assemblies through the supporting wall clutch part.

4. The movable laminated biological culture device according to claim 1, wherein bottom ends of the supporting walls are connected to the bottom part around, and the culture space is formed by enclosure of the bottom part, the supporting walls matched with the bottom part and a previous bottom part above the bottom part, and the biological development supply pile assembly is connected with the supporting walls to supply at least one of moisture, air and power to the culture space.

5. The movable laminated biological culture device according to claim 1, wherein top ends of the supporting walls are connected to the bottom part around, and the culture space is formed by enclosure of the bottom part, the supporting walls matched with the bottom part and a next bottom part below the bottom part, and the biological development supply pile assembly is connected with the supporting walls to supply at least one of moisture, air and power to the culture space.

6. The movable laminated biological culture device according to claim 1, wherein middle parts of the supporting walls are connected to the bottom part around, and the culture space is formed by enclosure of the bottom part, the supporting walls matched with the bottom part, a previous bottom part above the bottom part and previous supporting walls above the supporting walls, and the biological development supply pile assembly is connected with the supporting walls or the previous supporting walls above the supporting walls to supply at least one of moisture, air and power to the culture space.

7. The movable laminated biological culture device according to claim 1, wherein a structure of the bottom part is rectangular, and four supporting walls comprise two oppositely arranged supporting walls extending upward along the bottom part and two oppositely arranged supporting walls extending downward along the bottom part; the culture space is formed by enclosure of the bottom part, the two supporting walls extending upward along the bottom part, a previous bottom part above the bottom part and two supporting walls extending downward from the previous bottom part, and the biological development supply pile assembly is connected with the supporting walls extending upward along the bottom part or the supporting walls extending downward along the bottom part.

8. The movable laminated biological culture device according to claim 1, wherein the structure of the bottom part is rectangular, and the supporting walls comprise two slideway walls arranged oppositely, wherein a top and a bottom of each of the slideway walls are respectively provided with a slide rail part and a sliding part matched with each other up and down, and an upper layer culture space assembly is capable of sliding away from a lower layer culture space assembly along the slide rail part or the sliding part on each of the slideway walls, and the biological development supply pile assembly is connected with one of the supporting walls on at least one of four sides of the bottom part.

9. The movable laminated biological culture device according to claim 8, wherein other two supporting walls arranged vertically opposite to the two slideway walls are harvesting walls, and a length of each of the harvesting walls extending downwards along the bottom part is less than that of each of the slideway walls extending downwards along the bottom part, so that the upper layer culture space assembly slides away from the lower layer culture space assembly along the slide rail part or the sliding part on each of the slideway walls, meanwhile preset positions of organisms cultured in the lower layer culture space assembly are harvested and pushed away in a same direction by means of one of the harvesting walls of the upper layer culture space assembly, so as to realize harvesting of the organisms, and the biological development supply pile assembly is connected with at least one of the supporting walls of each of the slideway walls and/or each of the harvesting walls.

10. The movable laminated biological culture device according to claim 1, wherein the structure of the bottom part is rectangular, the supporting walls comprises two slideway walls arranged oppositely, a top and a bottom of each of the slideway walls are respectively provided with a slide rail part and a sliding part matched with each other up and down, and upper and lower parts of the bottom part corresponding to the slideway walls are respectively provided with a slide rail part and a sliding part matched with each of the slideway walls up and down, the bottom part is capable of sliding away from lower supporting walls along the slide rail part or the sliding part on each of the slideway walls, and the supporting walls are capable of sliding away from a lower bottom part along the slide rail part or the sliding part on the bottom part, and the biological development supply pile assembly is connected with one of the supporting walls on at least one of four sides of the bottom part.

11. The movable laminated biological culture device according to claim 1, wherein the bottom part comprises a plurality of pits arranged downwards, the pits are used for bearing nutrient substrates needed for cultivating preset organisms, so that the preset organisms absorb nutrients from the nutrient substrates in the pits, and the biological development supply pile assembly is also used for supplying at least one of moisture and air to the nutrient substrates in the pits.

12. A using method for movable laminated biological culture devices, comprising movable laminated biological culture devices and buildings according to claim 1, wherein each of the buildings comprises a beam-column part, and a supply pile connection part is arranged on the beam-column part, and each of the movable laminated biological culture devices is arranged in corresponding one of the buildings for use, and the biological development supply pile assembly is arranged at a position close to the supply pile connection part; the biological development supply pile assembly is connected or separated from the beam-column part through the supply pile connection part; before the culture space assembly is activated, the biological development supply pile assembly is connected with the beam-column part through the supply pile connection part; further, at least one of the preset water source, the preset gas source and the preset power supply is connected with the beam-column part and is connected with the biological development supply pile assembly along the beam-column part; and wherein the using method at least comprises:
when at least one of moisture, gas, or power needs to be supplied to culture space of each of the movable laminated biological culture devices, separating the biological development supply pile assembly from the beam-column part, and connecting each of the pile frame clutch parts of the biological development supply pile assembly with the culture space assembly through the supporting wall clutch part;
when the culture space assembly of each of the movable laminated biological culture devices needs to be transported and moved, separating each of the pile frame clutch parts of the biological development supply pile assembly from the supporting wall clutch part, so that the biological development supply pile assembly is separated from the culture space assembly, and then connecting the biological development supply pile assembly with the beam-column part through the supply pile connection part;
wherein the movable laminated biological culture devices are used for cultivation of organisms, and a process from sowing biological seeds to harvesting organisms at least comprises:
S1, biological culture process
S11, sowing biological seeds to a bottom part of an upper part first-layer culture space assembly to be seeded in each of the movable laminated biological culture devices, and then suspending a seeded culture space assembly upward to be separated from the upper part second-layer culture space assembly and then transferring to a preset bracket, so as to become a lower part first-layer culture space assembly, being about to enter the biological culture process, of the preset bracket;
S12, continuously sowing biological seeds to a bottom part of an upper part second-layer culture space assembly to be seeded in each of the movable laminated biological culture devices, and then suspending a seeded culture space assembly upward to be separated from an upper part third-layer culture space assembly, and then transferring to above a lower part first-layer culture space assembly, being about to enter the biological culture process, of the preset bracket, so as to become a lower part second-layer culture space assembly, being about to enter the biological culture process, of the preset bracket;
S13, continuously sowing biological seeds to a bottom part of an upper part third-layer culture space assembly to be seeded in each of the movable laminated biological culture devices, then suspending a seeded culture space assembly upward to be separated from an upper part fourth-layer culture space assembly, and then transferring to above a lower part second-layer culture space assembly, being about to enter the biological culture process, of the preset bracket, so as to become a lower part third-layer culture space assembly, being about to enter the biological culture process, of the preset bracket;
S14, by analogy, until completing sowing biological seeds of each layer of culture space assembly, and connecting the biological development supply pile assembly with a seeded culture space assembly in each layer on the preset bracket, so as to perform cultivation of organisms, until the cultivation of organisms is completed, and entering harvesting organisms process;
S2, harvesting organisms process
S21, separating the biological development supply pile assembly from a culture space assembly being completed cultivation of organisms on each layer of the preset bracket;
S22, harvesting organisms in an upper part first-layer culture space assembly to be harvested in each of the movable laminated biological culture devices on the preset bracket, and then suspending a harvested culture space assembly upward to be separated from the upper part second-layer culture space assembly and transferring to a preset device;
S23, continuously harvesting organisms in an upper part second-layer culture space assembly to be harvested of each of the movable laminated biological culture devices, and then suspending a harvested culture space assembly upward to be separated from the upper part third-layer of the culture space assembly, and then transferring to a preset device;
S24, continuously harvesting organisms in an upper part third-layer culture space assembly to be harvested in each of the movable laminated biological culture devices, and then suspending a harvested culture space assembly upward to be separated from an upper part fourth-layer culture space assembly and transferring to a preset device;
S25, by analogy, until completing harvesting of organisms in each layer of culture space assembly; or
a process from sowing biological seeds to harvesting organisms at least comprises:
S1, biological culture process
S11, sowing biological seeds to a bottom part of an upper part first-layer culture space assembly to be seeded in each of the movable laminated biological culture devices, and then pushing and transferring an upper part first-layer culture space assembly having been seeded to the preset bracket by means of the slide rail part and the sliding part on each of the slideway walls, to become a lower part first-layer culture space assembly, being about to enter the biological culture process, of the preset bracket;
S12, continuously sowing biological seeds to a bottom part of an upper part second-layer culture space assembly to be seeded in each of the movable laminated biological culture devices, and then continuously pushing and transferring an upper part second-layer culture space assembly having been seeded to above a lower part first-layer culture space assembly, being about to enter the biological culture process, of the preset bracket by means of the slide rail part and the sliding part on each of the slideway walls, so as to become a lower part second-layer culture space assembly, being about to enter the biological culture process, of the preset bracket;
S13, continuously sowing biological seeds to a bottom part of an upper part third-layer culture space assembly to be seeded in each of the movable laminated biological culture devices, and then continuously pushing and transferring an upper part third-layer culture space assembly having been seeded to above a lower part second-layer culture space assembly, being about to enter the biological culture process, of the preset bracket by means of the slide rail part and sliding part on each of the slideway walls, so as to become a lower part third-layer culture space assembly, being about to enter the biological culture process, of the preset bracket;
S14, by analogy, until completing sowing biological seeds of each layer of culture space assembly, and connecting the biological development supply pile assembly with a seeded culture space assembly in each layer on the preset bracket, so as to perform cultivation of organisms until the cultivation of organisms is completed, and entering harvesting organisms process;
S2, harvesting organisms process
S21, separating the biological development supply pile assembly from a culture space assembly being completed cultivation of organisms on each layer of the preset bracket;
S22, harvesting organisms in an upper part first-layer culture space assembly to be harvested in each of the movable laminated biological culture devices on the preset bracket, then pushing and transferring a harvested upper part first-layer culture space assembly to a preset device by means of the slide rail part and the sliding part on each of the slideway walls;
S23, continuously harvesting organisms in an upper part second-layer culture space assembly to be harvested of each of the movable laminated biological culture devices, and then continuously pushing and transferring a harvested upper part second-layer culture space assembly to a preset device by means of the slide rail part and sliding part on each of the slideway walls;
S24, continuously harvesting organisms in an upper part third-layer culture space assembly to be harvested in each of the movable laminated biological culture devices, and then continuously pushing and transferring a harvested upper part third-layer culture space assembly to a preset device by means of the slide rail part and sliding part on each of the slideway walls;
S25, by analogy, until completing harvesting of organisms in each layer of culture space assembly.

13. A using method for movable laminated biological culture devices, comprising movable laminated biological culture devices according to claim 1, wherein applying the movable laminated biological culture devices to a joint operation of harvesting organisms to sowing biological seeds at least comprises:
S1, separating the biological development supply pile assembly from a culture space assembly of each layer having been completed cultivation of organisms;
S2, harvesting organisms in an upper part first-layer culture space assembly to be harvested in each of the movable laminated biological culture devices, then purifying a bottom part of a first-layer culture space assembly having completed harvesting organisms and sowing biological seeds, and transferring a seeded culture space assembly to a preset bracket to become a lower part first-layer culture space assembly, being about to enter the biological culture process again, of the preset bracket;
S3, harvesting organisms in an upper part second-layer culture space assembly to be harvested in each of the movable laminated biological culture devices, then purifying a bottom part of a second-layer culture space assembly having completed harvesting organisms and sowing biological seeds, and transferring a seeded culture space assembly to an upper part of a lower part first-layer culture space assembly, being about to enter the biological culture process again, of the preset bracket, so as to become the lower part second-layer culture space assembly, being about to enter the biological culture process again, of the preset bracket;
S4, harvesting organisms in an upper part third-layer culture space assembly to be harvested in each of the movable laminated biological culture devices, then purifying a bottom part of a third-layer culture space assembly having completed harvesting organisms and sowing biological seeds, and transferring a seeded culture space assembly to an upper part of a lower part second-layer culture space assembly, being about to enter the biological culture process again, of the preset bracket, so as to become a lower part third-layer culture space assembly, being about to enter the biological culture process again, of the preset bracket;
S5, by analogy, simultaneously completing harvesting organisms, purification treatment and sowing biological seeds for each layer of culture space assembly through joint operation, and connecting the biological development supply pile assembly with a culture space assembly having been seeded in each layer on the preset bracket, so as to continue cultivation of organisms until the cultivation of organisms is completed, and then re-circulating into a joint operation process of harvesting organisms, synchronous purification treatment and sowing;
wherein applying the movable laminated biological culture devices to product mushroom at least comprises:
S1, separating the biological development supply pile assembly in each of the movable laminated biological culture devices from each layer of culture space assembly;
S2, separating a bottom part and supporting walls in an upper part first-layer culture space assembly in each of the movable laminated biological culture devices, simultaneously arranging mushroom culture substrate on an upper part first-layer bottom part, and transferring a bottom part bearing the mushroom culture substrate to a preset bracket to become a lower part first-layer bottom part, being about to enter a substrate disinfection process, of the preset bracket;
S3, separating a bottom part and supporting walls in an upper part second-layer culture space assembly in each of the movable laminated biological culture devices, and simultaneously arranging the mushroom culture substrate on an upper part second-layer bottom part, and transferring a bottom part bearing the mushroom culture substrate to a lower part first-layer bottom part of the preset bracket to become a lower part second-layer bottom part, being about to enter the substrate disinfection process, of the preset bracket;
S4, separating a bottom part and supporting walls in an upper part third-layer culture space assembly in each of the movable laminated biological culture devices, and simultaneously arranging the mushroom culture substrate on an upper part third-layer bottom part, and transferring a bottom part bearing the mushroom culture substrate to a lower part second-layer bottom part on the preset bracket to become a lower part third-layer bottom part, being about to enter the substrate disinfection process, of the preset bracket;
S5, by analogy, separating a bottom part and supporting walls in each layer of culture space assembly in each of the movable laminated biological culture devices, and completing arrangement of mushroom culture substrate on each layer of a bottom part, and overlapping a bottom part bearing the mushroom culture substrate, of each layer on a preset bracket;
S6, sterilizing a bottom part of each layer on the preset bracket together with bore mushroom culture substrate;
S7, performing mushroom inoculation on mushroom culture substrate on an upper part first-layer bottom part having been disinfected on the preset bracket, transferring a bottom part having been completed mushroom inoculation to a new preset bracket to become a lower part first-layer bottom part of the new preset bracket, and simultaneously arranging one of the supporting walls on the lower part first-layer bottom part to form lower part first-layer culture space of the new preset bracket, and arranging the biological development factor supply part in the lower part first-layer culture space together with the supporting wall;
S8, performing mushroom inoculation on an upper part second-layer bottom part having been disinfected on the preset bracket, transferring a bottom part having been inoculated with mushroom to a supporting wall on the lower part first-layer bottom part of the new preset bracket, and simultaneously arranging another supporting wall on the lower part first-layer bottom part to form a lower part second-layer culture space of the new preset bracket, and arranging the biological development factor supply part in the lower part second-layer culture space together with the supporting wall;
S9, performing mushroom inoculation on an upper part third-layer bottom part having been disinfected on the preset bracket, transferring a bottom part having been inoculated with mushroom to a supporting wall on a lower part second-layer bottom part of the new preset bracket, and simultaneously arranging another supporting wall on the lower part second-layer bottom part to form a lower part third-layer culture space of the new preset bracket, and arranging the biological development factor supply part in the lower part third-layer culture space together with the supporting wall;
S10, by analogy, until completing inoculation of mushroom culture substrate on a bottom part of each layer, and re-forming laminated culture space, and then connecting the biological development supply pile assembly with a culture space assembly of each layer, and entering mushroom culture process until mushroom culture is successful, and entering mushroom harvesting process;
S11, separating the biological development supply pile assembly from each layer of culture space assembly;
S12, harvesting mushroom in each culture space at an upper part of the new preset bracket, and separating supporting walls and a bottom part of each layer for later use.
